(19) United States
(12) Patent Application Publication
(10) Pub. No.: US 2011/0319803 A1
(43) Pub. Date: Dec. 29, 2011

(54) BANDAGING SYSTEM

(76) Inventor: Jennifer DiGrazia, Brooklyn, NY (US)

(21) Appl. No.: 13/004,866

(22) Filed: Jan. 11, 2011

Related U.S. Application Data
(63) Continuation-in-part of application No. 12/826,644, filed on Jun. 29, 2010.
(60) Provisional application No. 61/360,873, filed on Jul. 1, 2010.

(51) Int. Cl. A61F 13/02; A61F 13/00

(52) U.S. Cl. 602/43; 602/54

(57) ABSTRACT

A bandage includes a body portion and a gauze pad, the body portion being shaped, sized and configured depending upon the wound covering application, the gauze pad having three or four primary corners, the primary corners of the gauze pad in most instances being oriented towards edges of the body portion or length or width tangents of the body portion, preferably mid-points or mid-sections of the length or width tangents or body portion edges.

### CROSS-REFERENCE TO RELATED APPLICATIONS

This application claim priority under 35 U.S.C. §119(e) from co-pending U.S. Provisional Patent Application No. 61/360,873 by DiGrazia, entitled “Bandage”, filed Jul. 1, 2010, and is hereby incorporated by reference into the current application for all purposes. The application is also a continuation-in-part and claims priority from U.S. patent application Ser. No. 12/826,644 by DiGrazia, entitled “Wound and Bandage Protection System”, filed Jun. 29, 2010, and is hereby incorporated by reference into the current application for all purposes.

### FIELD OF THE INVENTION

The invention generally relates to bandages, more specifically to a bandage protection system that is primarily focused on covering wounds on joints and other areas of the body where it is difficult to maintain proper and comfortable coverage of wounds.

### BACKGROUND OF THE INVENTION

Throughout the history of bandage making, a common problem has plagued the adhesive bandage industry. In order to properly protect a wound, it should be covered and insulated from outside infectants. However, most adhesive bandages do not adequately protect a wound when applied. Makers of older bandages tried to size the gauze pad to allow for a thin strip of adhesive around the gauze pad to adhere to the skin around the wound. However, the strip of adhesive around the gauze pad would often buckle or come loose altogether, and not keep the wound properly sealed, and possibly cause discomfort. Recently companies have tried other solutions. Band-Aid® brand has given up on sealing the wound and has extended the gauze to the edge of the adhesive to maximize the amount of gauze available to cover the wound. Nexcare® has created bandages with extremely small gauze in relation to the bandage, allowing for a better seal, but providing less gauze in the exchange. Furthermore, these bandages tend not to perform well on joints, where the areas of adhesive do not conform to the bending of the limbs without causing a large amount of buckling of the gauze. Therefore, there is a need for a bandaging system to prevent buckling and loosening of the adhesive around the gauze pad of an adhesive bandage, particularly with regard to application of bandages to joints, while at the same time maximizing the amount of gauze available to cover the wound.

### SUMMARY OF THE INVENTION

In one aspect, the present invention features a bandage that includes a body portion and a gauze pad with four primary corners, in which at least two of the primary corners are oriented towards tangent lines of the body portion. In another aspect, the present invention features a bandage that includes a body portion and a gauze pad with four primary corners, in which sides of the gauze pad defined by the primary corners are not parallel to tangent lines of the body portion.

Embodiments of these two aspects may include one or more of the following. Embodiments may have a gauze pad shaped as a square, rhombus, kite or parallelogram. Embodiments may have a gauze pad that is positioned on a wound-facing side of the body portion in such manner that each of the corners of the gauze pad is oriented towards a different length tangent or width tangent of the body portion. Embodiments may have the gauze pad sized and positioned so as to define or substantially define four equal quadrants on a non-gauze portion of a wound facing side of the body portion. Embodiments may have a gauze pad with four sides where the gauze pad is sized and positioned so as to define or substantially define two equal quadrants on a non-gauze portion of the wound facing side of the body portion, with two sides of the four sides of the gauze pad defining an edge of a first quadrant of the two equal quadrants and another two sides of the four sides of the gauze pad defining an edge of a second quadrant of the two equal quadrants. Embodiments may have two opposing primary corners, each oriented toward a mid-section of a tangent line of the body portion. Embodiments may have all four primary corners oriented toward a mid-section of a tangent line of the body portion. Embodiments may have a center point of the gauze pad coinciding with a center point of the body portion. Embodiments may have one primary corner, two primary corners, two opposing primary corners, three primary corners or all four primary corners extend to the edges of the body portion. Embodiments may have the gauze pad oriented in such manner that a ray from each of the gauze pad's primary corners intersects a tangent of the body portion at an angle of 55°, 65°, or 90° or at any angle within the range of 55°-90° or preferably between 65°-90°. Embodiments may have adhesive on at least a portion of a wound facing side of the body portion. Embodiments may have a body portion that includes self adherent material.

Embodiments of these two aspects may also include one or more of the following. Embodiments may have the body portion configured with a central portion and two legs that extend in a lengthwise direction from the central portion toward the same width tangent. In such embodiments, the shape of the body portion may be defined by two parallel arcs or parabolas that are connected to each other at respective proximate ends that are distal to the central portion. Embodiments may have the body portion configured with a central portion that has a uniform or substantially uniform width defined by flattened top and bottom edges and two side regions extending from opposing sides of the central portion to opposing ends of the body portion with the width of the side regions narrowing as the side regions extend from the central portion to the ends of the body portion. Embodiments may have a body portion shape that has a central almond or oval shaped portion and bulbous side portions extending from opposing sides of the central almond or oval shaped portion. Embodiments may have the body portion configured in a square or rectangular, almond or oval shape. Embodiments may have the body portion configured with rounded corners. Embodiments may have the body portion configured with a head portion and a tail portion. In such embodiments, the head portion may be configured as a truncated oval, diamond, or almond shape with the tail portion flaring out with an increasing width as it extends lengthwise from a first side of the head portion forming, in conjunction with the head portion, a concavity along top and bottom edges of the body portion. Embodiments may have the body portion configured with a first end region that extends to a first end of the body portion and a second end region with a smaller width than the first end region that extends to a second end of the body portion. In such embodiments, the body portion width may taper or remain the same as the body portion extends from the first end region to the second end region. In such embodiments, the second side region may have a greater length than the first side region and a top edge and a bottom edge of the body portion may each be configured as a continuous convex curve. Embodiments may have the body portion configured with a central portion and oval shaped side regions that extend from opposing sides of the central portion. In such embodiments, the central portion may have a uniform or substantially uniform width defined by flattened top and bottom edges. In such embodiments, the body portion may have narrow portions that connect the central portion to each of the oval shaped side regions. In such embodiments, the central portion may have concave top and bottom edges, resulting in a widening width from the middle of the central portion to the oval shaped end portions.

Embodiments of these two aspects may also have the body portion configured as a bowtie with a central narrow portion that has a narrowed width and two side regions. The two side regions, in the bowtie configured body portion, may each have a first side and a second side with the first sides of the two side regions on opposing adjacent sides of the central narrow portion and the second sides of the two side regions on opposing ends of the body portion. Each of the side regions may have a first side region portion that extends from the first side of the side region toward the second side of the side region, where a width of the first side region portion of each of the side regions widens with respect to the width of the central narrow portion as it extends from the first side of the side regions toward the second side of the side regions. Such embodiments may also have a second side region portion for each of the side regions that extends from the first side region portion to the second side of the side regions where the width of the second side region portion narrows as it extends from the first side region portion to the second side of the side region. Such embodiments with a bowtie configured body portion may also have the primary corners of the gauze pad that are oriented toward length tangents of the body portion positioned within the central narrow portion. Such embodiments with a bowtie configured body portion may also be configured so that midpoint of the central narrow portion does not correspond with a midpoint of the length tangents of the body portion. Such embodiments with a bowtie configured body portion may also be configured so that the edges at opposing ends of the body portion taper the length of the body portion as the edges extend from corners of the body portion toward an inflection point. Such embodiments with a bowtie configured body portion may also have the body portion edge of each of the side regions defined by a single circular or elliptical arc. Such embodiments with a bowtie configured body portion may, alternatively, have an edge of each of the side regions defined by two circular or elliptical arcs that extend from opposing sides of the central narrow portion and meet at an inflection point.

In another aspect, the present invention features a bandage that includes a body portion and a gauze pad with four primary corners, in which sides of the gauze pad defined by the primary corners are parallel to tangent lines of the body portion. Embodiments may have the body portion configured with a head portion and a tail portion. In such embodiments, the head portion may be configured as a truncated oval, diamond, or almond shape with the tail portion flaring out with an increasing width as it extends lengthwise from a first side of the head portion forming, in conjunction with the head portion, a concavity along top and bottom edges of the body portion. Embodiments may have a square or rectangular gauze pad. Embodiments may have the body portion configured in a bowtie shape.

In another aspect, the present invention features a bandage that includes a body portion and a gauze pad with three primary corners. Embodiments may include a body portion that is triangular with each of the primary corners oriented toward a different edge of the body portion. Such embodiments may have the gauze pad oriented on the wound-facing side of the body portion in such manner that each of the primary corners of the gauze pad is oriented toward a mid-point or a mid-section of a different edge of the body portion. Embodiments may also include a body portion that is circular shaped. Embodiments may have adhesive on at least a portion of a wound facing side of the body portion.

In one exemplary embodiment of a bandage according to the present invention, the bandage has a body portion and a gauze pad on a wound-facing side of the body portion. The gauze pad is shaped and positioned on the body portion in such manner that the bandage is configured as a “diamond gauze” bandage. The body portion has adhesive on at least a portion of a wound facing side or, alternatively, the body portion is comprised of self adherent material.

In another exemplary embodiment of a bandage according to the present invention, the bandage is a “diamond gauze” bandage that has a body portion and a gauze pad. The body portion has adhesive on at least a portion of a wound facing side or, alternatively, the body portion is comprised of self adherent material. The gauze pad is shaped as a square, rhombus or parallelogram and oriented on the wound-facing side of the body portion in such manner that each of the corners of the gauze pad are oriented towards a different length tangent or width tangent of the body portion.

In another exemplary embodiment of a bandage according to the present invention, the bandage is a “triangular gauze” bandage that has a triangular shaped body portion and a triangular shaped gauze pad. The triangular shaped body portion has adhesive on at least a portion of a wound facing side. The triangular shaped gauze pad is oriented on the wound-facing side of the body portion in such manner that each of the corners of the gauze pad is oriented toward a different edge of the body portion. The triangular shaped gauze pad may be oriented on the wound-facing side of the body portion in such manner that each of the corners of the gauze pad is oriented toward a mid-point or a mid-section of a different edge of the body portion.

In another exemplary embodiment of a bandage according to the present invention, the bandage is a “triangular gauze” bandage that has a circular shaped body portion and a triangular shaped gauze pad. The circular shaped body portion has adhesive on at least a portion of a wound facing side. The triangular shaped gauze pad is provided on the wound-facing side of the body portion.

In addition, the invention also relates to a kit that includes all or a subset of the bandages disclosed herein. These and other embodiments may have one or more of the following advantages. These embodiments may resolve one or more issues of durability, comfort and ease of application that are not adequately resolved by current bandages.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 12(a) shows a bottom wound-facing side of an exemplary embodiment of a “diamond gauze” bandage according to the present invention;

FIG. 12(b) shows a top non-wound-facing side of the exemplary embodiment of the “diamond gauze” bandage illustrated in FIG. 12(a);

FIG. 12(c) shows a side view along a length of the exemplary embodiment of the “diamond gauze” bandage illustrated in FIGS. 12(a) and (b) (the view from the opposing length side is a mirror image);

FIG. 12(d) shows a side view along a width of the exemplary embodiment of the “diamond gauze” bandage illustrated in FIGS. 12(a) and (b) (the view from the opposing width side is a minor image);

FIG. 12(e) shows a perspective view of the exemplary embodiment of the “diamond gauze” bandage illustrated in FIGS. 12(a) and (b);

FIG. 12(f) shows a second perspective view of the exemplary embodiment of the “diamond gauze” bandage illustrated in FIGS. 12(a) and (b);

FIG. 13(a) shows a bottom wound-facing side of an exemplary embodiment of a “diamond gauze” bandage according to the present invention;

FIG. 13(b) shows a top non-wound-facing side of the exemplary embodiment of the “diamond gauze” bandage illustrated in FIG. 13(a);

FIG. 13(c) shows a side view along a length of the exemplary embodiment of the “diamond gauze” bandage illustrated in FIGS. 13(a) and (b) (the view from the opposing length side is a mirror image);

FIG. 13(d) shows a side view along a width of the exemplary embodiment of the “diamond gauze” bandage illustrated in FIGS. 13(a) and (b) (the view from the opposing width side is a minor image);

FIG. 13(e) shows a perspective view of the exemplary embodiment of the “diamond gauze” bandage illustrated in FIGS. 13(a) and (b);

FIG. 13(f) shows a second perspective view of the exemplary embodiment of the “diamond gauze” bandage illustrated in FIGS. 13(a) and (b);

FIG. 14(a) shows a bottom wound-facing side of an exemplary embodiment of a “diamond gauze” bandage according to the present invention;

FIG. 14(b) shows a top non-wound-facing side of the exemplary embodiment of the “diamond gauze” bandage illustrated in FIG. 14(a);

FIG. 14(c) shows a side view along a length of the exemplary embodiment of the “diamond gauze” bandage illustrated in FIGS. 14(a) and (b) (the view from the opposing length side is a mirror image);

FIG. 14(d) shows a side view along a width of the exemplary embodiment of the “diamond gauze” bandage illustrated in FIGS. 14(a) and (b) (the view from the opposing width side is a minor image);

FIG. 14(e) shows a perspective view of the exemplary embodiment of the “diamond gauze” bandage illustrated in FIGS. 14(a) and (b);

FIG. 15(a) shows a bottom wound-facing side of an exemplary embodiment of a “diamond gauze” bandage according to the present invention;

FIG. 15(b) shows a top non-wound-facing side of the exemplary embodiment of the “diamond gauze” bandage illustrated in FIG. 15(a);

FIG. 15(c) shows a side view along a length of the exemplary embodiment of the “diamond gauze” bandage illustrated in FIGS. 15(a) and (b) (the view from the opposing length side is a mirror image);

FIG. 15(d) shows a side view along a width of the exemplary embodiment of the “diamond gauze” bandage illustrated in FIGS. 15(a) and (b) (the view from the opposing width side is a minor image);

FIG. 15(e) shows a perspective view of the exemplary embodiment of the “diamond gauze” bandage illustrated in FIGS. 15(a) and (b);

FIG. 16(a) shows a bottom wound-facing side of an exemplary embodiment of a “diamond gauze” bandage according to the present invention;

FIG. 16(b) shows a top non-wound-facing side of the exemplary embodiment of the “diamond gauze” bandage illustrated in FIG. 16(a);

FIG. 16(c) shows a side view along a length of the exemplary embodiment of the “diamond gauze” bandage illustrated in FIGS. 16(a) and (b) (the view from the opposing length side is a mirror image);

FIG. 16(d) shows a side view along a width of the exemplary embodiment of the “diamond gauze” bandage illustrated in FIGS. 16(a) and (b) (the view from the opposing width side is a minor image);

FIG. 16(e) shows a perspective view of the exemplary embodiment of the “diamond gauze” bandage illustrated in FIGS. 16(a) and (b);

FIG. 17(a) shows a bottom wound-facing side of an exemplary embodiment of a “diamond gauze” bandage according to the present invention;

FIG. 17(b) shows a top non-wound-facing side of the exemplary embodiment of the “diamond gauze” bandage illustrated in FIG. 17(a);

FIG. 17(c) shows a side view along a width of the exemplary embodiment of the “diamond gauze” bandage illustrated in FIGS. 17(a) and (b) (the view from the opposing width side is a minor image);

FIG. 17(d) shows a side view along a length of the exemplary embodiment of the “diamond gauze” bandage illustrated in FIGS. 17(a) and (b) (the view from the opposing length side is a mirror image);

FIG. 17(e) shows a perspective view of the exemplary embodiment of the “diamond gauze” bandage illustrated in FIGS. 17(a) and (b);

FIG. 18(a) shows a bottom wound-facing side of an exemplary embodiment of a “diamond gauze” bandage according to the present invention;

FIG. 18(b) shows a top non-wound-facing side of the exemplary embodiment of the “diamond gauze” bandage illustrated in FIG. 18(a);

FIG. 18(c) shows a side view along a length of the exemplary embodiment of the “diamond gauze” bandage illustrated in FIGS. 18(a) and (b) (the view from the opposing length side is a mirror image);

FIG. 18(d) shows a side view along a width of the exemplary embodiment of the “diamond gauze” bandage illustrated in FIGS. 18(a) and (b) (the view from the opposing width side is a minor image);

FIG. 18(e) shows a perspective view of the exemplary embodiment of the “diamond gauze” bandage illustrated in FIGS. 18(a) and (b);

FIG. 19(a) shows a bottom wound-facing side of an exemplary embodiment of a “diamond gauze” bandage according to the present invention;

FIG. 19(b) shows a top non-wound-facing side of the exemplary embodiment of the “diamond gauze” bandage illustrated in FIG. 19(a);

FIG. 19(c) shows a first side view along a width tangent 199D of the exemplary embodiment of the “diamond gauze” bandage illustrated in FIGS. 19(a) and (b);

FIG. 19(d) shows a second side view along a length tangent 199C of the exemplary embodiment of the “diamond gauze” bandage illustrated in FIGS. 19(a) and (b);

FIG. 19(e) shows a third side view along a width tangent 199B of the exemplary embodiment of the “diamond gauze” bandage illustrated in FIGS. 19(a) and (b);

FIG. 19(f) shows a fourth side view along a length tangent 199A of the exemplary embodiment of the “diamond gauze” bandage illustrated in FIGS. 19(a) and (b);

FIG. 19(g) shows a perspective view of the exemplary embodiment of the “diamond gauze” bandage illustrated in FIGS. 19(a) and (b);

FIG. 20(a) shows a bottom wound-facing side of an exemplary embodiment of a “diamond gauze” bandage according to the present invention;

FIG. 20(b) shows a top non-wound-facing side of the exemplary embodiment of the “diamond gauze” bandage illustrated in FIG. 20(a);

FIG. 20(c) shows a first side view along a length tangent 209C of the exemplary embodiment of the “diamond gauze” bandage illustrated in FIGS. 20(a) and (b);

FIG. 20(d) shows a second side view along a width tangent 209B of the exemplary embodiment of the “diamond gauze” bandage illustrated in FIGS. 20(a) and (b);

FIG. 20(e) shows a third side view along a length tangent 209A of the exemplary embodiment of the “diamond gauze” bandage illustrated in FIGS. 20(a) and (b);

FIG. 20(f) shows a fourth side view along a width tangent 209D of the exemplary embodiment of the “diamond gauze” bandage illustrated in FIGS. 20(a) and (b);

FIG. 20(g) shows a perspective view of the exemplary embodiment of the “diamond gauze” bandage illustrated in FIGS. 20(a) and (b);

FIG. 21(a) shows a bottom wound-facing side of an exemplary embodiment of a “diamond gauze” bandage according to the present invention;

FIG. 21(b) shows a top non-wound-facing side of the exemplary embodiment of the “diamond gauze” bandage illustrated in FIG. 21(a);

FIG. 21(c) shows a side view along a length of the exemplary embodiment of the “diamond gauze” bandage illustrated in FIGS. 21(a) and (b) (the view from the opposing length side is a mirror image);

FIG. 21(d) shows a side view along a width of the exemplary embodiment of the “diamond gauze” bandage illustrated in FIGS. 21(a) and (b) (the view from the opposing width side is a minor image);

FIG. 21(e) shows a perspective view of the exemplary embodiment of the “diamond gauze” bandage illustrated in FIGS. 21(a) and (b);

FIG. 22(a) shows a bottom wound-facing side of an exemplary embodiment of a “triangular gauze” bandage according to the present invention;

FIG. 22(b) shows a top non-wound-facing side of the exemplary embodiment of the “triangular gauze” bandage illustrated in FIG. 22(a);

FIG. 22(c) shows a first side view along a length of the exemplary embodiment of the “triangular gauze” bandage illustrated in FIGS. 22(a) and (b);

FIG. 22(d) shows a second side view along a length of the exemplary embodiment of the “triangular gauze” bandage illustrated in FIGS. 22(a) and (b);

FIG. 22(e) shows a third side view along a width of the exemplary embodiment of the “triangular gauze” bandage illustrated in FIGS. 22(a) and (b);

FIG. 22(f) shows a fourth side view along a width of the exemplary embodiment of the “triangular gauze” bandage illustrated in FIGS. 22(a) and (b);

FIG. 22(g) shows a perspective view of the exemplary embodiment of the “triangular gauze” bandage illustrated in FIGS. 22(a) and (b);

FIG. 23(a) shows a bottom wound-facing side of an exemplary embodiment of a “triangular gauze” bandage according to the present invention;

FIG. 23(b) shows a top non-wound-facing side of the exemplary embodiment of the “triangular gauze” bandage illustrated in FIG. 23(a);

FIG. 23(c) shows a first side view along a length of the exemplary embodiment of the “triangular gauze” bandage illustrated in FIGS. 23(a) and (b);

FIG. 23(d) shows a second side view along a width of the exemplary embodiment of the “triangular gauze” bandage illustrated in FIGS. 23(a) and (b);

FIG. 23(e) shows a third side view along a length of the exemplary embodiment of the “triangular gauze” bandage illustrated in FIGS. 23(a) and (b);

FIG. 23(f) shows a fourth side view along a width of the exemplary embodiment of the “triangular gauze” bandage illustrated in FIGS. 23(a) and (b);

FIG. 23(g) shows a perspective view of the exemplary embodiment of the “triangular gauze” bandage illustrated in FIGS. 23(a) and (b);

FIG. 24(a) shows a bottom wound-facing side of an exemplary embodiment of a “diamond gauze” bandage according to the present invention;

FIG. 24(b) shows a perspective view of the exemplary embodiment of the “diamond gauze” bandage illustrated in FIGS. 24(a) and (c);

FIG. 24(c) shows a top non-wound-facing side of the exemplary embodiment of the “diamond gauze” bandage illustrated in FIG. 24(a);

FIG. 24(d) shows a side view along a length tangent 249C of the exemplary embodiment of the “diamond gauze” bandage illustrated in FIGS. 24(a) and (b) (the view from the opposing length tangent 249A is a mirror image);

FIG. 24(e) shows a side view along a width tangent 249B of the exemplary embodiment of the “diamond gauze” bandage illustrated in FIGS. 24(a) and (b);

FIG. 24(f) shows a side view along a width tangent 249D of the exemplary embodiment of the “diamond gauze” bandage illustrated in FIGS. 24(a) and (b);

FIG. 25(a) shows a top view of an exemplary gauze pad configuration with concave sides for a “diamond gauze” bandage according to the present invention;

FIG. 25(b) shows a top view of an exemplary gauze pad configuration with convex sides for a “diamond gauze” bandage according to the present invention;

FIG. 25(c) shows a top view of an exemplary gauze pad configuration with undulating sides for a “diamond gauze” bandage according to the present invention;

FIG. 25(d) shows a top view of an exemplary gauze pad configuration with rounded corners for a “diamond gauze” bandage according to the present invention;

FIG. 25(e) shows a top view of an exemplary gauze pad configuration with a square shape for a “diamond gauze” bandage according to the present invention;

FIG. 25(f) shows a top view of an exemplary gauze pad configuration with an offset diamond shape for a “diamond gauze” bandage according to the present invention;

FIG. 25(g) shows a top view of an exemplary gauze pad configuration with a diamond or rhombus shape for a “diamond gauze” bandage according to the present invention;

FIG. 25(h) shows a top view of an exemplary gauze pad configuration with a kite shape for a “diamond gauze” bandage according to the present invention;

FIG. 25(i) shows a top view of an exemplary gauze pad configuration with a rectangular shape for a “diamond gauze” bandage according to the present invention;

FIG. 25(j) shows a top view of an exemplary gauze pad configuration with two opposing corners cut off for a “diamond gauze” bandage according to the present invention;

FIG. 25(k) shows a top view of an exemplary gauze pad configuration with four cut off corners for a “diamond gauze” bandage according to the present invention;

FIG. 25(l) shows a definition of length and width in terms of a horizontal axis and a vertical axis for a “diamond gauze” bandage according to the present invention;

FIG. 25(m) illustrates an exemplary preferred range of gauze pad orientation for a “diamond gauze” bandage according to the present invention;

FIG. 26(a) shows a bottom wound-facing side of an exemplary embodiment of a “diamond gauze” bandage according to the present invention;

FIG. 26(b) shows a top non-wound-facing side of the exemplary embodiment of the “diamond gauze” bandage illustrated in FIG. 26(a);

FIG. 26(c) shows a side view along a length tangent 269C of the exemplary embodiment of the “diamond gauze” bandage illustrated in FIGS. 26(a) and (b) (the view from the opposing length tangent 269A is a mirror image);

FIG. 26(d) shows a side view along a width tangent 269B of the exemplary embodiment of the “diamond gauze” bandage illustrated in FIGS. 26(a) and (b);

FIG. 26(e) shows a side view along a width tangent 269D of the exemplary embodiment of the “diamond gauze” bandage illustrated in FIGS. 26(a) and (b);

FIG. 26(f) shows a perspective view of the exemplary embodiment of the “diamond gauze” bandage illustrated in FIGS. 26(a) and (b);

FIG. 27(a) shows a bottom wound-facing side of an exemplary embodiment of a “diamond gauze” bandage according to the present invention;

FIG. 27(b) shows a top non-wound-facing side of the exemplary embodiment of the “diamond gauze” bandage illustrated in FIG. 27(a);

FIG. 27(c) shows a side view along a length tangent 279A of the exemplary embodiment of the “diamond gauze” bandage illustrated in FIGS. 27(a) and (b) (the view from the opposing length tangent 279C is a minor image);

FIG. 27(d) shows a side view along a width tangent line 279B of the exemplary embodiment of the “diamond gauze” bandage illustrated in FIGS. 27(a) and (b) (the view from the opposing width tangent 279D is a mirror image);

FIG. 27(e) shows a perspective view of the exemplary embodiment of the “diamond gauze” bandage illustrated in FIGS. 27(a) and (b);

FIG. 28(a) shows a bottom wound-facing side of an exemplary embodiment of a “diamond gauze” bandage according to the present invention;

FIG. 28(b) shows a top non-wound-facing side of the exemplary embodiment of the “diamond gauze” bandage illustrated in FIG. 28(a);

FIG. 28(c) shows a side view along a length tangent 289A of the exemplary embodiment of the “diamond gauze” bandage illustrated in FIGS. 28(a) and (b) (the view from the opposing length tangent 289C is a minor image);

FIG. 28(d) shows a side view along a width tangent 289B of the exemplary embodiment of the “diamond gauze” bandage illustrated in FIGS. 28(a) and (b) (the view from the opposing width tangent 289D is a mirror image);

FIG. 28(e) shows a perspective view of the exemplary embodiment of the “diamond gauze” bandage illustrated in FIGS. 28(a) and (b);

FIG. 29(a) shows a bottom wound-facing side of an exemplary embodiment of a “diamond gauze” bandage according to the present invention;

FIG. 29(b) shows a top non-wound-facing side of the exemplary embodiment of the “diamond gauze” bandage illustrated in FIG. 29(a);

FIG. 29(c) shows a side view along a length tangent 299C of the exemplary embodiment of the “diamond gauze” bandage illustrated in FIGS. 29(a) and (b) (the view from the opposing length tangent 299A is a minor image);

FIG. 29(d) shows a side view along a width tangent 299D of the exemplary embodiment of the “diamond gauze” bandage illustrated in FIGS. 29(a) and (b);

FIG. 29(e) shows a side view along a width tangent line 299B of the exemplary embodiment of the “diamond gauze” bandage illustrated in FIGS. 29(a) and (b);

FIG. 29(f) shows a perspective view of the exemplary embodiment of the “diamond gauze” bandage illustrated in FIGS. 29(a) and (b);

FIG. 30(a) shows a bottom wound-facing side of an exemplary embodiment of a “diamond gauze” bandage according to the present invention;

FIG. 30(b) shows a top non-wound-facing side of the exemplary embodiment of the “diamond gauze” bandage illustrated in FIG. 30(a);

FIG. 30(c) shows a side view along a length tangent 309A of the exemplary embodiment of the “diamond gauze” bandage illustrated in FIGS. 30(a) and (b) (the view from the opposing tangent 309C is a mirror image);

FIG. 30(d) shows a side view along a width tangent 309B of the exemplary embodiment of the “diamond gauze” bandage illustrated in FIGS. 30(a) and (b) (the view from the opposing width tangent 309D is a mirror image);

FIG. 30(e) shows a perspective view of the exemplary embodiment of the “diamond gauze” bandage illustrated in FIGS. 30(a) and (b);

FIG. 31(a) shows a bottom wound-facing side of an exemplary embodiment of a “diamond gauze” bandage according to the present invention;

FIG. 31(b) shows a top non-wound-facing side of the exemplary embodiment of the “diamond gauze” bandage illustrated in FIG. 31(a);

FIG. 31(c) shows a side view along a length tangent 319A of the exemplary embodiment of the “diamond gauze” bandage illustrated in FIGS. 31(a) and (b) (the view from the opposing length tangent 319C is a minor image);

FIG. 31(d) shows a side view along a width tangent 319B of the exemplary embodiment of the “diamond gauze” bandage illustrated in FIGS. 31(a) and (b) (the view from the opposing width tangent 319D is a mirror image);

FIG. 31(e) shows a perspective view of the exemplary embodiment of the “diamond gauze” bandage illustrated in FIGS. 31(a) and (b);

FIG. 32(a) shows a bottom wound-facing side of an exemplary embodiment of a “diamond gauze” bandage according to the present invention;

FIG. 32(b) shows a top non-wound-facing side of the exemplary embodiment of the “diamond gauze” bandage illustrated in FIG. 32(a);

FIG. 32(c) shows a side view along a length tangent 329A of the exemplary embodiment of the “diamond gauze” bandage illustrated in FIGS. 32(a) and (b) (the view from the opposing length tangent 329C is a minor image);

FIG. 32(d) shows a side view along a width tangent 329B of the exemplary embodiment of the “diamond gauze” bandage illustrated in FIGS. 32(a) and (b) (the view from the opposing width tangent 329D is a mirror image);

FIG. 32(e) shows a perspective view of the exemplary embodiment of the “diamond gauze” bandage illustrated in FIGS. 32(a) and (b);

FIG. 33(a) shows a bottom wound-facing side of an exemplary embodiment of a “diamond gauze” bandage according to the present invention;

FIG. 33(b) shows a top non-wound-facing side of the exemplary embodiment of the “diamond gauze” bandage illustrated in FIG. 33(a);

FIG. 33(c) shows a side view along a length tangent 339A of the exemplary embodiment of the “diamond gauze” bandage illustrated in FIGS. 33(a) and (b) (the view from the opposing length tangent 339C is a minor image);

FIG. 33(d) shows a side view along a width tangent 339B of the exemplary embodiment of the “diamond gauze” bandage illustrated in FIGS. 33(a) and (b) (the view from the opposing width tangent 339D is a mirror image);

FIG. 33(e) shows a perspective view of the exemplary embodiment of the “diamond gauze” bandage illustrated in FIGS. 33(a) and (b);

FIG. 34(a) shows a bottom wound-facing side of an exemplary embodiment of a “diamond gauze” bandage according to the present invention;

FIG. 34(b) shows a top non-wound-facing side of the exemplary embodiment of the “diamond gauze” bandage illustrated in FIG. 34(a);

FIG. 34(c) shows a side view along a length tangent 349A of the exemplary embodiment of the “diamond gauze” bandage illustrated in FIGS. 34(a) and (b) (the view from the opposing length tangent 349C is a minor image);

FIG. 34(d) shows a side view along a width tangent 349B of the exemplary embodiment of the “diamond gauze” bandage illustrated in FIGS. 34(a) and (b) (the view from the opposing tangent 349D is a minor image);

FIG. 34(e) shows a perspective view of the exemplary embodiment of the “diamond gauze” bandage illustrated in FIGS. 34(a) and (b);

FIG. 35(a) shows a bottom wound-facing side of an exemplary embodiment of a “diamond gauze” bandage according to the present invention;

FIG. 35(b) shows a top non-wound-facing side of the exemplary embodiment of the “diamond gauze” bandage illustrated in FIG. 35(a);

FIG. 35(c) shows a side view along a length tangent 359A of the exemplary embodiment of the “diamond gauze” bandage illustrated in FIGS. 35(a) and (b) (the view from the opposing length tangent 359C is a minor image);

FIG. 35(d) shows a side view along a width tangent 359B of the exemplary embodiment of the “diamond gauze” bandage illustrated in FIGS. 35(a) and (b) (the view from the opposing width tangent 359D is a mirror image);

FIG. 35(e) shows a perspective view of the exemplary embodiment of the “diamond gauze” bandage illustrated in FIGS. 35(a) and (b);

FIG. 36(a) shows a bottom wound-facing side of an exemplary embodiment of a “diamond gauze” bandage according to the present invention;

FIG. 36(b) shows a top non-wound-facing side of the exemplary embodiment of the “diamond gauze” bandage illustrated in FIG. 36(a);

FIG. 36(c) shows a side view along a length tangent 369A of the exemplary embodiment of the “diamond gauze” bandage illustrated in FIGS. 36(a) and (b) (the view from the opposing length tangent 369C is a minor image);

FIG. 36(d) shows a side view along a width tangent 369B of the exemplary embodiment of the “diamond gauze” bandage illustrated in FIGS. 36(a) and (b) (the view from the opposing width tangent 369D is a mirror image);

FIG. 36(e) shows a perspective view of the exemplary embodiment of the “diamond gauze” bandage illustrated in FIGS. 36(a) and (b);

FIG. 37(a) shows a bottom wound-facing side of an exemplary embodiment of a “diamond gauze” bandage according to the present invention;

FIG. 37(b) shows a top non-wound-facing side of the exemplary embodiment of the “diamond gauze” bandage illustrated in FIG. 37(a);

FIG. 37(c) shows a side view along a length tangent 379A of the exemplary embodiment of the “diamond gauze” bandage illustrated in FIGS. 37(a) and (b) (the view from the opposing length tangent 379C is a minor image);

FIG. 37(d) shows a side view along a width tangent 379B of the exemplary embodiment of the “diamond gauze” bandage illustrated in FIGS. 37(a) and (b) (the view from the opposing width tangent 379D is a mirror image);

FIG. 37(e) shows a perspective view of the exemplary embodiment of the “diamond gauze” bandage illustrated in FIGS. 37(a) and (b);

FIG. 38(a) shows a bottom wound-facing side of an exemplary embodiment of a “diamond gauze” bandage according to the present invention;

FIG. 38(b) shows a top non-wound-facing side of the exemplary embodiment of the “diamond gauze” bandage illustrated in FIG. 38(a);

FIG. 38(c) shows a side view along a length tangent 389A of the exemplary embodiment of the “diamond gauze” bandage illustrated in FIGS. 38(a) and (b) (the view from the opposing length tangent 389C is a minor image);

FIG. 38(d) shows a side view along a width tangent 389B of the exemplary embodiment of the “diamond gauze” bandage illustrated in FIGS. 38(a) and (b) (the view from the opposing width tangent 389D is a mirror image);

FIG. 38(e) shows a perspective view of the exemplary embodiment of the “diamond gauze” bandage illustrated in FIGS. 38(a) and (b);

FIG. 39(a) shows a bottom wound-facing side of an exemplary embodiment of a “diamond gauze” bandage according to the present invention;

FIG. 39(b) shows a top non-wound-facing side of the exemplary embodiment of the “diamond gauze” bandage illustrated in FIG. 39(a);

FIG. 39(c) shows a side view along a length tangent 399A of the exemplary embodiment of the “diamond gauze” bandage illustrated in FIGS. 39(a) and (b) (the view from the opposing length tangent 399C is a minor image);

FIG. 39(d) shows a side view along a width tangent 399B of the exemplary embodiment of the “diamond gauze” bandage illustrated in FIGS. 39(a) and (b) (the view from the opposing width tangent 399D is a minor image);

FIG. 39(e) shows a perspective view of the exemplary embodiment of the “diamond gauze” bandage illustrated in FIGS. 39(a) and (b);

FIG. 40(a) shows a bottom wound-facing side of an exemplary embodiment of a “diamond gauze” bandage according to the present invention;

FIG. 40(b) shows a top non-wound-facing side of the exemplary embodiment of the “diamond gauze” bandage illustrated in FIG. 40(a);

FIG. 40(c) shows a side view along a length tangent 409A of the exemplary embodiment of the “diamond gauze” bandage illustrated in FIGS. 40(a) and (b) (the view from the opposing length tangent 409C is a minor image);

FIG. 40(d) shows a side view along a width tangent 409B of the exemplary embodiment of the “diamond gauze” bandage illustrated in FIGS. 40(a) and (b) (the view from the opposing width tangent 409D is a mirror image);

FIG. 40(e) shows a perspective view of the exemplary embodiment of the “diamond gauze” bandage illustrated in FIGS. 40(a) and (b);

FIG. 41(a) shows a bottom wound-facing side of an exemplary embodiment of a “diamond gauze” bandage according to the present invention;

FIG. 41(b) shows an exploded bottom wound-facing side of an exemplary embodiment of a “diamond gauze” bandage according to the present invention;

FIG. 41(c) shows a top non-wound-facing side of the exemplary embodiment of the “diamond gauze” bandage illustrated in FIG. 41(a);

FIG. 41(d) shows a side view along a length tangent 419A of the exemplary embodiment of the “diamond gauze” bandage illustrated in FIGS. 41(a) and (b) (the view from the opposing length tangent 419C is a minor image);

FIG. 41(e) shows a side view along a width tangent 419B of the exemplary embodiment of the “diamond gauze” bandage illustrated in FIGS. 41(a) and (b) (the view from the opposing width tangent 419D is a mirror image);

FIG. 41(f) shows a perspective view of the exemplary embodiment of the “diamond gauze” bandage illustrated in FIGS. 41(a) and (b);

FIG. 42(a) shows a bottom wound-facing side of an exemplary embodiment of a “diamond gauze” bandage according to the present invention;

FIG. 42(b) shows a top non-wound-facing side of the exemplary embodiment of the “diamond gauze” bandage illustrated in FIG. 42(a);

FIG. 42(c) shows a side view along a length tangent 429A of the exemplary embodiment of the “diamond gauze” bandage illustrated in FIGS. 42(a) and (b) (the view from the opposing length tangent 429C is a minor image);

FIG. 42(d) shows a side view along a width tangent 429B of the exemplary embodiment of the “diamond gauze” bandage illustrated in FIGS. 42(a) and (b) (the view from the opposing width tangent 429D is a mirror image);

FIG. 42(e) shows a perspective view of the exemplary embodiment of the “diamond gauze” bandage illustrated in FIGS. 42(a) and (b);

FIG. 43(a) shows a bottom wound-facing side of an exemplary embodiment of a “diamond gauze” bandage according to the present invention;

FIG. 43(b) shows a top non-wound-facing side of the exemplary embodiment of the “diamond gauze” bandage illustrated in FIG. 43(a);

FIG. 43(c) shows a side view along a length tangent 439A of the exemplary embodiment of the “diamond gauze” bandage illustrated in FIGS. 43(a) and (b) (the view from the opposing length tangent 439C is a minor image);

FIG. 43(d) shows a side view along a width tangent 439B of the exemplary embodiment of the “diamond gauze” bandage illustrated in FIGS. 43(a) and (b) (the view from the opposing width tangent 439D is a mirror image);

FIG. 43(e) shows a perspective view of the exemplary embodiment of the “diamond gauze” bandage illustrated in FIGS. 43(a) and (b);

FIG. 44(a) shows a bottom wound-facing side of an exemplary embodiment of a “diamond gauze” bandage according to the present invention;

FIG. 44(b) shows a top non-wound-facing side of the exemplary embodiment of the “diamond gauze” bandage illustrated in FIG. 44(a);

FIG. 44(c) shows a side view along a length tangent 449A of the exemplary embodiment of the “diamond gauze” bandage illustrated in FIGS. 44(a) and (b) (the view from the opposing length tangent 449C is a minor image);

FIG. 44(d) shows a side view along a width tangent 449B of the exemplary embodiment of the “diamond gauze” bandage illustrated in FIGS. 44(a) and (b) (the view from the opposing width tangent 449D is a minor image);

FIG. 44(e) shows a perspective view of the exemplary embodiment of the “diamond gauze” bandage illustrated in FIGS. 44(a) and (b);

FIG. 45(a) shows a bottom wound-facing side of an exemplary embodiment of a “diamond gauze” bandage according to the present invention;

FIG. 45(b) shows a top non-wound-facing side of the exemplary embodiment of the “diamond gauze” bandage illustrated in FIG. 45(a);

FIG. 45(c) shows a side view along a length tangent 459A of the exemplary embodiment of the “diamond gauze” bandage illustrated in FIGS. 45(a) and (b) (the view from the opposing length tangent 459C is a minor image);

FIG. 45(d) shows a side view along a width tangent 459B of the exemplary embodiment of the “diamond gauze” bandage illustrated in FIGS. 45(a) and (b) (the view from the opposing width tangent 459D is a mirror image);

FIG. 45(e) shows a perspective view of the exemplary embodiment of the “diamond gauze” bandage illustrated in FIGS. 45(a) and (b);

FIG. 46(a) shows a bottom wound-facing side of an exemplary embodiment of a “diamond gauze” bandage according to the present invention;

FIG. 46(b) shows a top non-wound-facing side of the exemplary embodiment of the “diamond gauze” bandage illustrated in FIG. 46(a);

FIG. 46(c) shows a side view along a length tangent 469A of the exemplary embodiment of the “diamond gauze” bandage illustrated in FIGS. 46(a) and (b) (the view from the opposing length tangent 469C, as well as from width tangents 469B and 469D are identical);

FIG. 46(d) shows a perspective view of the exemplary embodiment of the “diamond gauze” bandage illustrated in FIGS. 46(a) and (b);

FIG. 47(a) shows a bottom wound-facing side of an exemplary embodiment of a “diamond gauze” bandage according to the present invention;

FIG. 47(b) shows a top non-wound-facing side of the exemplary embodiment of the “diamond gauze” bandage illustrated in FIG. 47(a);

FIG. 47(c) shows a side view along a length tangent 479A of the exemplary embodiment of the “diamond gauze” bandage illustrated in FIGS. 47(a) and (b) (the view from the opposing length tangent 479C is a minor image);

FIG. 47(d) shows a side view along a width tangent 479B of the exemplary embodiment of the “diamond gauze” bandage illustrated in FIGS. 47(a) and (b) (the view from the opposing width tangent 479D is a mirror image);

FIG. 47(e) shows a perspective view of the exemplary embodiment of the “diamond gauze” bandage illustrated in FIGS. 47(a) and (b);

FIG. 48(a) shows a bottom wound-facing side of an exemplary embodiment of a “diamond gauze” bandage according to the present invention;

FIG. 48(b) shows a top non-wound-facing side of the exemplary embodiment of the “diamond gauze” bandage illustrated in FIG. 48(a);

FIG. 48(c) shows a side view along a length tangent 489A of the exemplary embodiment of the “diamond gauze” bandage illustrated in FIGS. 48(a) and (b) (the view from the opposing length tangent 489C is a minor image);

FIG. 48(d) shows a side view along a width tangent 489B of the exemplary embodiment of the “diamond gauze” bandage illustrated in FIGS. 48(a) and (b) (the view from the opposing width tangent 489D is a mirror image);

FIG. 48(e) shows a perspective view of the exemplary embodiment of the “diamond gauze” bandage illustrated in FIGS. 48(a) and (b);

FIG. 49(a) shows a bottom wound-facing side of an exemplary embodiment of a “diamond gauze” bandage according to the present invention;

FIG. 49(b) shows an exploded bottom wound-facing side of an exemplary embodiment of a “diamond gauze” bandage according to the present invention;

FIG. 49(c) shows a top non-wound-facing side of the exemplary embodiment of the “diamond gauze” bandage illustrated in FIG. 49(a);

FIG. 49(d) shows a side view along a length tangent 499A of the exemplary embodiment of the “diamond gauze” bandage illustrated in FIGS. 49(a) and (b) (the view from the length tangent line 499C, as well as the views from width tangents 499B and 499D are identical to the view from length tangent 499A);

FIG. 49(e) shows a perspective view of the exemplary embodiment of the “diamond gauze” bandage illustrated in FIGS. 49(a) and (b);

FIG. 50(a) shows a bottom wound-facing side of an exemplary embodiment of a “diamond gauze” bandage according to the present invention;

FIG. 50(b) shows a top non-wound-facing side of the exemplary embodiment of the “diamond gauze” bandage illustrated in FIG. 50(a);

FIG. 50(c) shows a side view along a length of the exemplary embodiment of the “diamond gauze” bandage illustrated in FIGS. 50(a) and (b) (the view from the opposing length side is a mirror image);

FIG. 50(d) shows a side view along a width of the exemplary embodiment of the “diamond gauze” bandage illustrated in FIGS. 50(a) and (b) (the view from the opposing width side is a minor image);

FIG. 50(e) shows a perspective view of the exemplary embodiment of the “diamond gauze” bandage illustrated in FIGS. 50(a) and (b);

FIG. 51(a) shows a bottom wound-facing side of an exemplary embodiment of a “diamond gauze” bandage according to the present invention;

FIG. 51(b) shows a top non-wound-facing side of the exemplary embodiment of the “diamond gauze” bandage illustrated in FIG. 51(a);

FIG. 51(c) shows a side view along a length tangent 519C of the exemplary embodiment of the “diamond gauze” bandage illustrated in FIGS. 51(a) and (b) (the view from the opposing length tangent 519A is a mirror image);

FIG. 51(d) shows a side view along a width tangent 519B of the exemplary embodiment of the “diamond gauze” bandage illustrated in FIGS. 51(a) and (b);

FIG. 51(e) shows a side view along a width tangent 519D of the exemplary embodiment of the “diamond gauze” bandage illustrated in FIGS. 51(a) and (b);

FIG. 51(f) shows a perspective view of the exemplary embodiment of the “diamond gauze” bandage illustrated in FIGS. 51(a) and (b);

FIG. 52(a) shows a bottom wound-facing side of an exemplary embodiment of a “diamond gauze” bandage according to the present invention;

FIG. 52(b) shows a top non-wound-facing side of the exemplary embodiment of the “diamond gauze” bandage illustrated in FIG. 52(a);

FIG. 52(c) shows a side view along a length tangent 529C of the exemplary embodiment of the “diamond gauze” bandage illustrated in FIGS. 52(a) and (b) (the view from the opposing length tangent 529A is a mirror image);

FIG. 52(d) shows a side view along a width tangent 529B of the exemplary embodiment of the “diamond gauze” bandage illustrated in FIGS. 52(a) and (b);

FIG. 52(e) shows a side view along a width tangent 529D of the exemplary embodiment of the “diamond gauze” bandage illustrated in FIGS. 52(a) and (b); and

FIG. 52(f) shows a perspective view of the exemplary embodiment of the “diamond gauze” bandage illustrated in FIGS. 52(a) and (b).

### DETAILED DESCRIPTION OF THE INVENTION

In certain situations, it may be necessary to apply a bandage to a wound, particularly on joints, whose constant movement may cause a gauze pad to move or buckle. In those situations, according to the present invention, a “diamond gauze” or “triangle gauze” bandage, as discussed below, may be found to have advantages over other bandages known in the art. After much experimentation, while designing related bandages, inventor found that the “diamond gauze” and “triangle gauze” bandages may provide superior performance over existing bandages, particularly with regard to a more reliable and comfortable fit as well as easier application to the wound, among other advantages. Furthermore, due to the configurations of the “diamond gauze” and “triangle gauze” bandages, they may in some cases provide superior adherence and conformability to a wound area with a maximal area of gauze, particularly for wounds on joints.

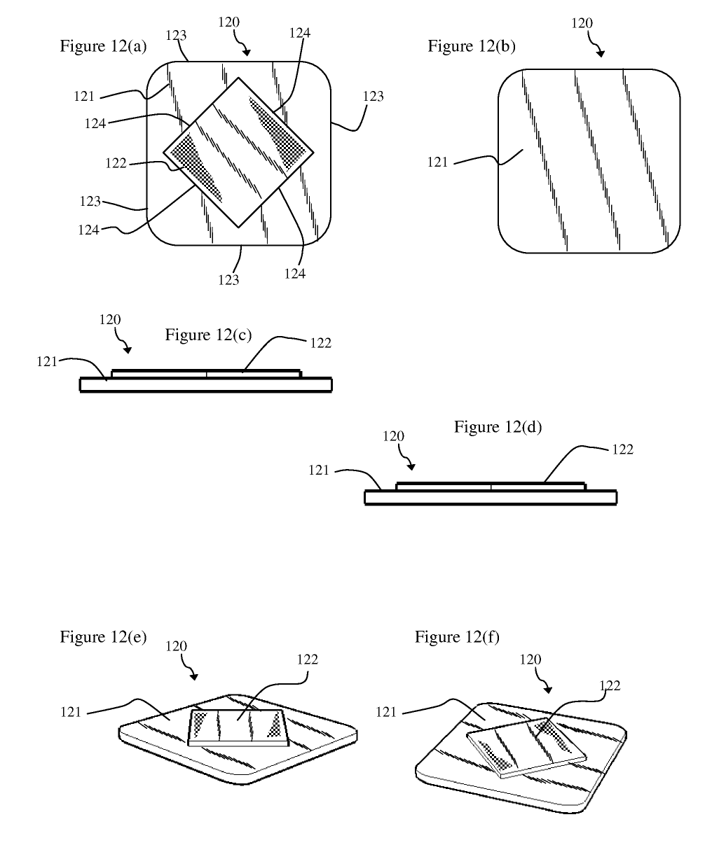

FIGS. 12(a)-12(f) are, respectively, a bottom wound-facing view, a top non-wound facing view, two side views and two perspective views of an exemplary embodiment of a “diamond gauze” bandage 120 according to the present invention. The “diamond gauze” bandage 120 has a body portion 121 in a square shape, preferably with rounded corners, which may be made of any suitable material, preferably a stretchable and breathable material, which is known in the art to be used for bandaging wounds.

A gauze pad 122 is attached to the wound-facing side of the body portion 121. In the context of this specification, gauze, or gauze pad, refers to any material or composite of material that may be therapeutically used as a pad over a wound. For example, the gauze pad may be made of cotton or a polyester blend fabric. The fabric may be covered with a plastic porous film such as Telfa® which prevents or minimizes wound adhesion. Furthermore, the gauze pad may be backed with a film that prevents body fluids from penetrating through the gauze pad and then through the body portion of the bandage.

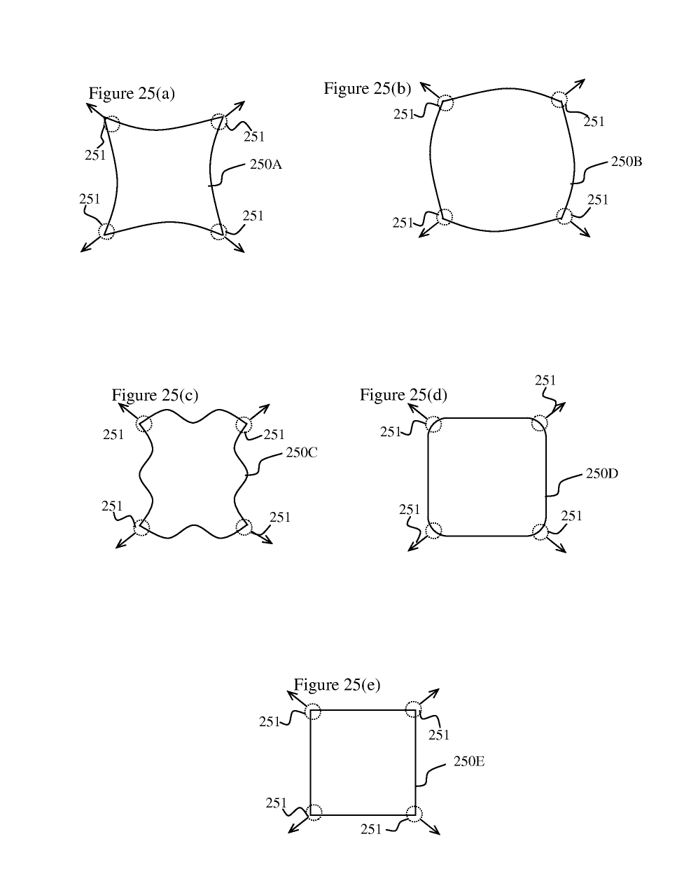

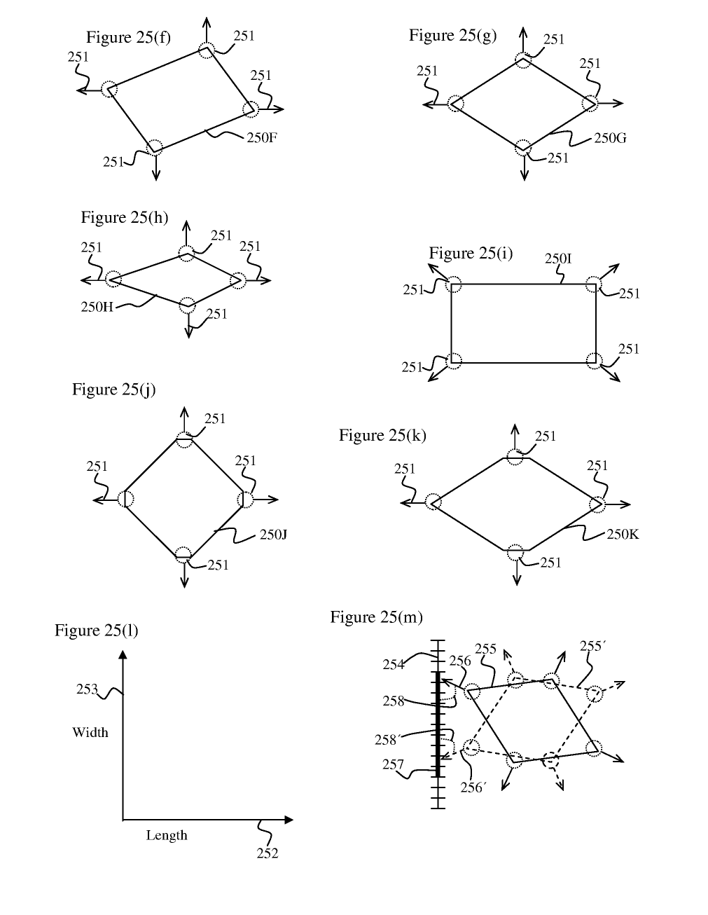

FIGS. 25(a)-(k) show several exemplary gauze pad configurations 250A, 250B, 250C, 250D, 250E, 250F, 250G, 250H, 250I, 250J, 250K for “diamond gauze” bandages according to the present invention. The gauze pad 122, as shown in FIGS. 12(a)-12(f), has a square shape equivalent to the gauze pad configuration 250E. Alternatively, the gauze pad 122 may be configured in the shape of a diamond or rhombus as exemplified by the gauze pad configuration 250G, in a “kite shape” (quadrilateral with two distinct pairs of equal adjacent sides) as exemplified by the gauze pad configuration 250H, a rectangle as exemplified by the gauze pad configuration 250I, or in the shape of an “offset diamond” (parallelogram with opposing sides of equal length, adjacent sides of unequal length and corner angles not equal to 90 degrees) as exemplified by the gauze pad configuration 250F. One or more corners of the gauze pad 122 may be rounded, as exemplified by the corners of gauze pad configuration 250D, or one or more corners of the gauze pad 122 may be cut off, as exemplified by corners of gauze pad configurations 250J and 250K. Furthermore, sides 124 of the gauze pad 122 need not be straight. For example, one or more of the sides 124 of the gauze pad 122 may be a convex or concave curve, as exemplified by the sides of gauze pad configurations 250A and 250B, respectively, or an undulating line, as exemplified by the sides of the gauze pad configuration 250C. Any combination of side and corner configurations, such as the exemplary ones shown herein, may be applied to the gauze pad 122. However, all possible shape configurations of a gauze pad in “diamond gauze” bandages should have four “primary corners”, i.e. the four corners of a quadrilateral or any four locations on a polygon where the polygon narrows and that would together approximate a quadrilateral with sides defined by sides of the gauze pad between the primary corners. In the exemplary gauze pad configurations 250A, 250B, 250C, 250D, 250E, 250F, 250G, 250H, 250I, 250J, 250K, there are four primary corners 251, identified by circles, and they are oriented as indicated by directional arrows associated with each of the primary corners 251.

The primary corners of the gauze pad 122 are offset from the corners of the body portion 121 such that the sides 124 of the gauze pad 122 are not parallel to edges 123 of the body portion 121. Preferably, the gauze pad 122 is oriented so that each of the sides of the gauze pad 122 is parallel to a hypotenuse of an isosceles right triangle formed with two adjacent edges 123 of the body portion 121.

In regard to the “diamond gauze” bandages disclosed herein, length and width tangents of a body portion may be defined by the edges of the body portion such as the edges 123 of the body portion 121. Thus, where a body portion as illustrated herein is square or rectangular, the length and width tangents are not explicitly defined as they are the edges of the body portion itself. Similarly, in exemplary embodiments of “diamond gauze” bandages where the body portion has a different shape but one of the length or width tangents is completely or almost completely contiguous with the body portion, the tangent line is not explicitly defined, such as for the “diamond gauze” bandage 120. Otherwise, the length and width tangents are a set of explicitly defined tangents that are tangential to the body portion edges and form a square or rectangular shape. For purposes of the discussion herein, length is defined along the horizontal axis and width is defined along the vertical axis, as shown in FIG. 25(l). Each of the primary corners of the gauze pad 122 is oriented towards a different length or width tangent of the body portion 121. Similarly, for other embodiments of a “diamond gauze” bandage, each of the primary corners is oriented towards a different length or width tangent of the body portion.

The gauze pad 122 may have the same center point as the body portion 121, and/or each of the primary corners of the gauze pad 122 may be oriented toward a mid-point or midsection of one of the edges 123, which in this case are essentially equivalent to the length or width tangents of the body portion 121. The midsection of a length or width tangent is a portion of the length or width tangent preferably equal to half of the length or width tangent and centered on the midpoint of the length or width tangent. More preferably, the midsection may be a third, a quarter, or an eighth, or another fraction of the length or width tangent that is less than half of the length or width tangent.

FIG. 25(m) illustrates an exemplary preferred range of gauze pad orientation for a “diamond gauze” bandage according to the present invention. Preferably, all primary corners of a gauze pad in a “diamond gauze” bandage should be oriented towards the midpoints of the length and width tangents. Of course, this is not always practical, given the varied shapes, functions, and other limiting factors of the specific bandage configurations. Line 254 represents an exemplary length or width tangent that is divided into equal sub-lengths defined by tick-marks, and a preferable orientation range 257 is indicated by the thicker portion of the line 254. A gauze pad 255 with four primary corners indicated by circles with orientations indicated by their associated arrows is positioned so that one of the primary corners is oriented towards an upper limit of the preferable orientation range 257, while a gauze pad 255′ is oriented so that one of the primary corners is oriented towards a lower limit of the preferable orientation range 257. The preferable orientation range 257 may represent the midsection of the line 254, as discussed above. Alternatively, the preferable range of gauze pad orientation may be defined by a minimal angle formed by the intersection of a length or width tangent and a ray that extends in the direction that a primary corner is oriented, where the angle is on the side of the ray that is proximate to the point where the ray would form a right angle with the length or width tangent. In FIG. 25(m), angles 258, 258′ are formed by the intersection of the line 254 and rays 256, 256′ that extend in the direction that the primary corner of gauze pads 255, 255′ are oriented. In this instance, the orientation range 257 of the line 254 is defined by a minimal value for the angles 258, 258′. The orientation range 257 in this instance is not necessarily centered on the midpoint of the line 254. The angles 258, 258′ preferably have a range from a minimal value of 55° to a maximal value of 90° at the right angled intersection of the line 254 and the rays 256, 256′. More preferably, the angles 258, 258′ have a minimal value of 65°, 75°, or 85°.

The size of the gauze pad 122 is such that the primary corners of the gauze pad 122 do not reach the edges 123 of the body portion 121. Alternatively, the gauze pad 122 may be sized shaped and/or positioned in such manner that one or more of the primary corners of the gauze pad reach the edges 123 of the body portion 121. The gauze pad 122 may be sized and positioned so as to substantially define four equal quadrants on the wound facing side of the body portion 121, i.e. each side of the gauze pad 132 defines the majority of one edge of one of the quadrants, the quadrants being areas of the wound facing side of the body portion 121 that are not covered by the gauze pad 122. The bottom wound-facing side of the body portion 121 may have adhesive as those used for bandages in the art, or alternatively, the body portion may include self adherent material, such as the nonwoven laminate used in 3M™ Coban™ Self-Adherent Wrap. The adhesive may be applied to all or part of the bottom wound-facing side of the body portion 121. The gauze pad 122 may or may not be affixed to the body portion 121 via the same adhesive as that which is used to affix the “diamond gauze” bandage 120 to a patient.

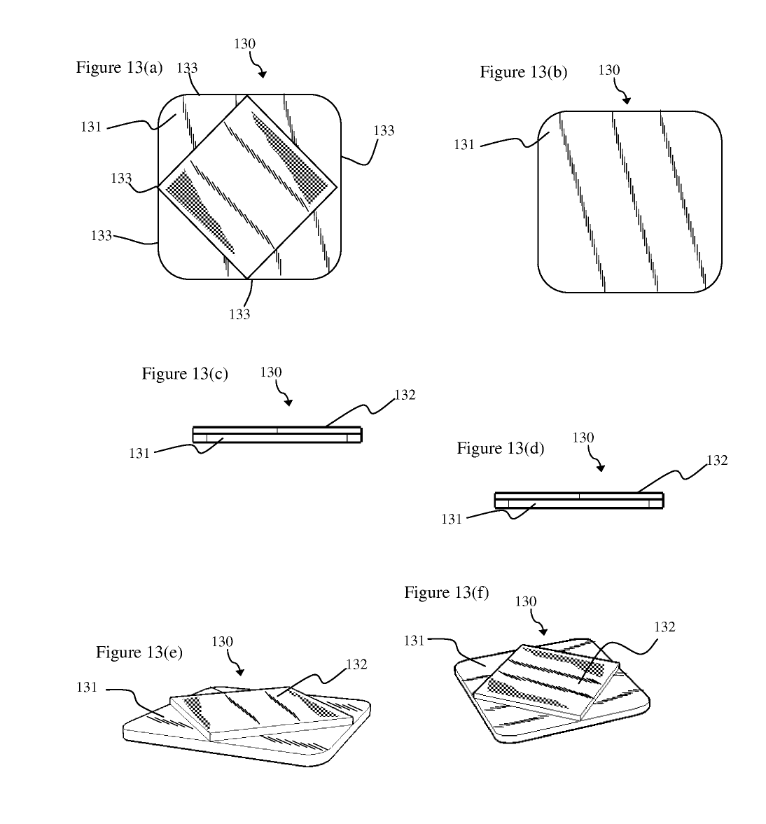

FIGS. 13(a)-13(f) are, respectively, a bottom wound-facing view, a top non-wound facing view, two side views and two perspective views of an exemplary embodiment of a “diamond gauze” bandage 130 according to the present invention. In contrast to “diamond gauze” bandage 120, the size of gauze pad 132 in the “diamond gauze” bandage 130 is such that the primary corners of the gauze pad 132 reach edges 133 of a body portion 131 of the “diamond gauze” bandage 130. The gauze pad 132 may be sized and positioned so as to define four equal quadrants on the wound facing side of the body portion 131. In an alternative exemplary embodiment, the gauze pad 132 may be sized and positioned so as to substantially define four equal quadrants on the wound facing side of the body portion 131. In all other respects, the configuration of the “diamond gauze” bandage 130 is comparable to the configuration of the “diamond gauze” bandage 120. Thus, the discussion above by “diamond gauze” bandage 120 regarding issues such as gauze pad shape, positioning and orientation as well as body portion materials, adhesive type and placement are applicable to the “diamond gauze” bandage 130 as well to other “diamond gauze” bandages, including the exemplary embodiments of a “diamond gauze” bandage discussed below.

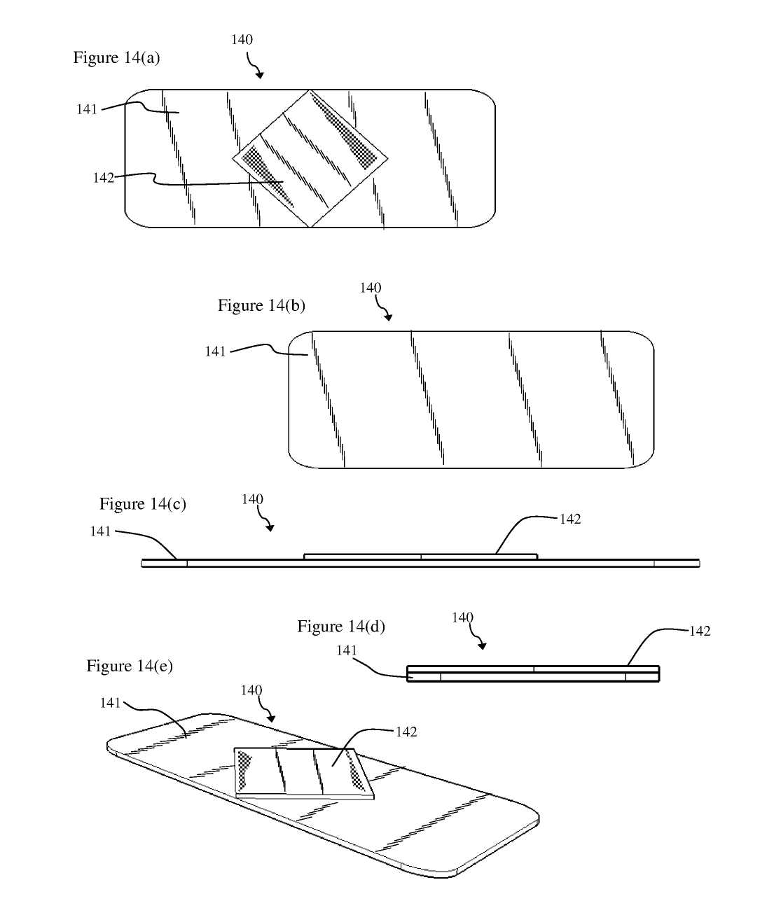

FIGS. 14(a) through 14(e) are, respectively, a bottom wound-facing view, a top non-wound facing view, two side views and one perspective view of an exemplary embodiment of a “diamond gauze” bandage 140 according to the present invention. In the “diamond gauze” bandage 140, a body portion 141 has a rectangular shape, preferably with rounded corners. A gauze pad 142 is configured in a square shape and is oriented with respect to the body portion 141 such that the sides of the gauze pad 142 are not parallel to the edges of the body portion 141. The gauze pad 142 may be sized so that two opposing primary corners may touch two opposing edges of the body portion 141 while the remaining primary corners of the gauze pad 142 do not touch the remaining edges of the body portion 141. Moreover, for any “diamond gauze” bandages, according to the present invention, the gauze pads may be sized so that two opposing primary corners may touch two opposing edges of the body portions. The gauze pad 142 may have the same center point as the body portion 141 and/or each of the primary corners of the gauze pad 142 may be oriented toward a mid-point of one of the edges of body portion 141.

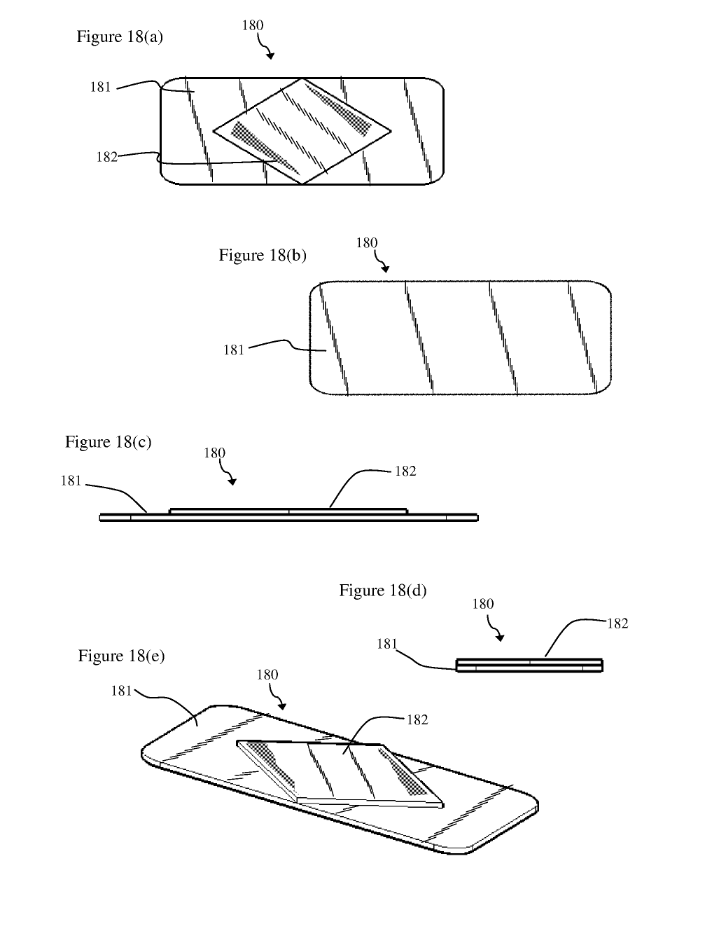

FIGS. 18(a) through 18(e) are, respectively, a bottom wound-facing view, a top non-wound facing view, two side views and one perspective view of an exemplary embodiment of a “diamond gauze” bandage 180 according to the present invention. In the “diamond gauze” bandage 180, a body portion 181 has a rectangular shape, preferably with rounded corners. A gauze pad 182 is configured in the shape of a diamond, with two opposing primary corners of the gauze pad 182 touching two opposing length edges of the body portion 181. In all other respects, the configuration of the “diamond gauze” bandage 180 is comparable to the configuration of the “diamond gauze” bandage 140.

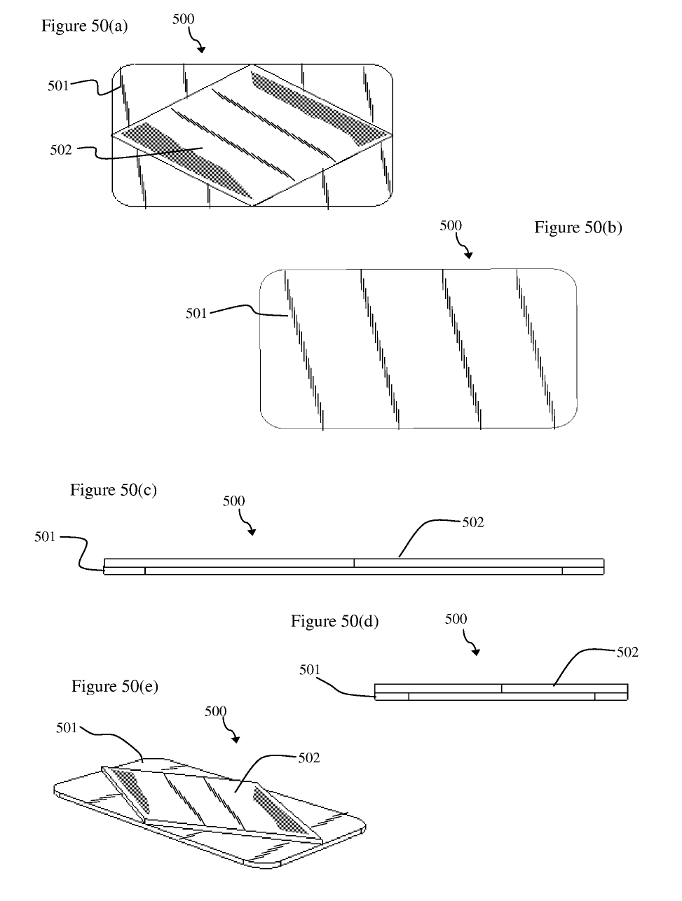

FIGS. 50(a) through 50(e) are, respectively, a bottom wound-facing view, a top non-wound facing view, two side views and one perspective view of an exemplary embodiment of a “diamond gauze” bandage 500 according to the present invention. In the “diamond gauze” bandage 500, a body portion 501 has a rectangular shape, preferably with rounded corners. A gauze pad 502 is configured in the shape of a diamond, with all four primary corners of the gauze pad 502 touching all four edges of the body portion 501. In all other respects, the configuration of the “diamond gauze” bandage 500 is comparable to the configuration of the “diamond gauze” bandage 180. Moreover, for any of the “diamond gauze” bandages, according to the present invention, the gauze pad may be sized and positioned so that some or all of the gauze pad corners extend to the edges of the body portion.

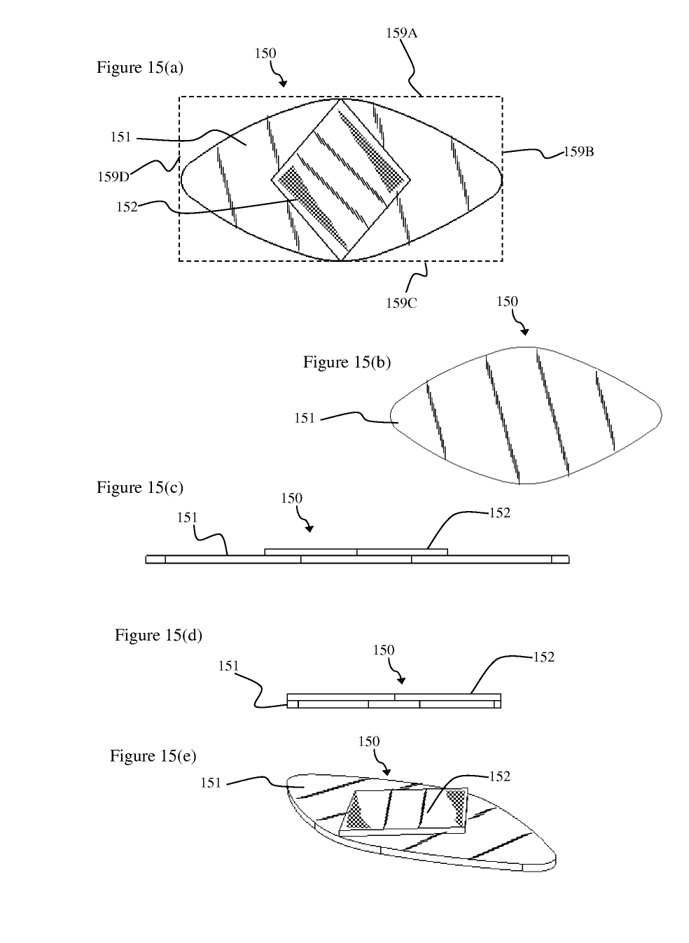

FIGS. 15(a)-15(e) are, respectively, a bottom wound-facing view, a top non-wound facing view, two side views and one perspective view of another exemplary embodiment of a “diamond gauze” bandage 150 according to the present invention. The body portion 151 of the “diamond gauze” bandage 150 is an “almond” shape which among other bandaging uses may be particularly useful for bandaging a finger tip or similar appendage. The gauze pad 152 is oriented with respect to the body portion 151 of the “diamond gauze” bandage 150 such that the sides of the gauze pad 152 are not parallel to length tangents 159A, 159C and width tangents 159B, 159D of the body portion 151. The gauze pad 152 may have the same center point as the body portion 151 and/or each of the corners of the gauze pad 152 may be oriented toward a mid-point of one of the length tangents 159A, 159C or width tangents 159B, 159D.

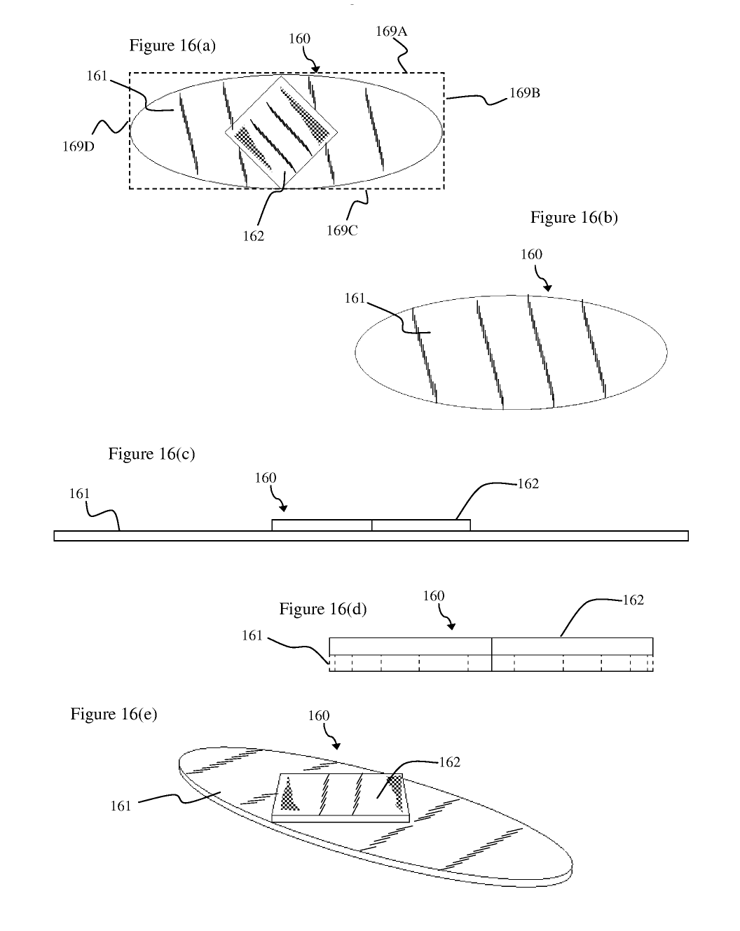

FIGS. 16(a)-16(e) are, respectively, a bottom wound-facing view, a top non-wound facing view, two side views and one perspective view of another exemplary embodiment of a “diamond gauze” bandage 160 according to the present invention. The body portion 161 of the “diamond gauze” bandage 160 is an oval shape which among other bandaging uses may be particularly useful for bandaging a finger tip or similar appendage. The gauze pad 162 is oriented with respect to the body portion 161 such that the sides of the gauze pad 162 are not parallel to length tangents 169A, 169C and width tangents 169B, 169D of the body portion 161. The gauze pad 162 may have the same center point as the body portion 161 and/or each of the corners of the gauze pad 162 may be oriented toward a mid-point of one of the length tangents 169A, 169C or width tangents 169B, 169D.

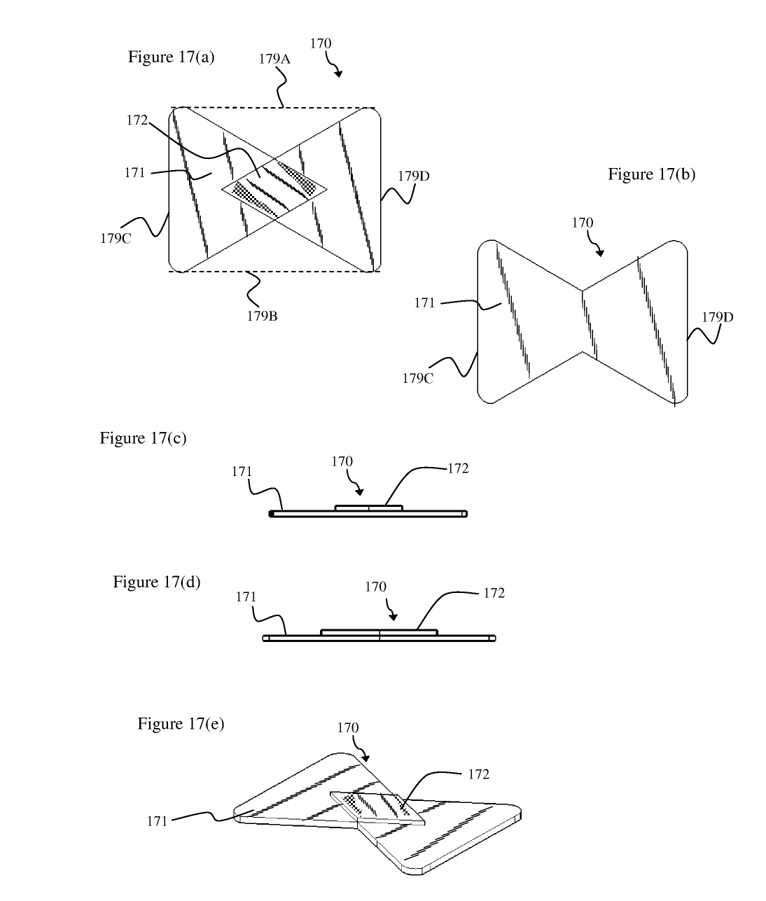

FIGS. 17(a)-17(e) are, respectively, a bottom wound-facing view, a top non-wound facing view, two side views and one perspective view of another exemplary embodiment of a “diamond gauze” bandage 170 according to the present invention. The body portion 171 of the “diamond gauze” bandage 170 is a “bowtie” or “hourglass” shape which among other bandaging uses may be particularly useful for bandaging a finger tip or similar appendage. The gauze pad 172 is oriented with respect to the body portion 171 of the “diamond gauze” bandage 170 such that the sides of the gauze pad 172 are not parallel to the length tangents 179A, 179B and edges 179C and 179D of the body portion 171, the edges 179C and 179D being essentially equivalent to width tangents in this exemplary embodiment. The gauze pad 172 may have the same center point as the body portion 171, and/or each of the corners of the gauze pad 172 may be oriented toward a mid-point of one of the tangents 179A and 179B and edges 179C and 179D of the body portion 171 of the “diamond gauze” bandage 170.

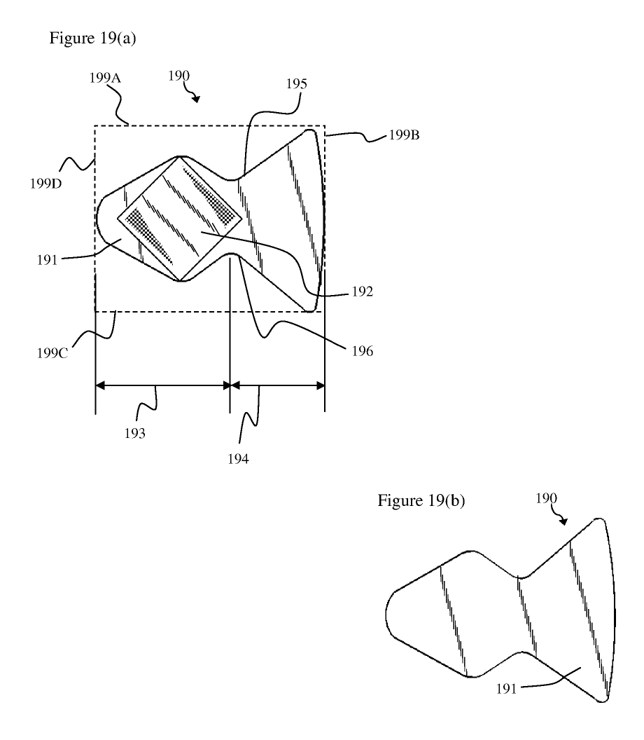

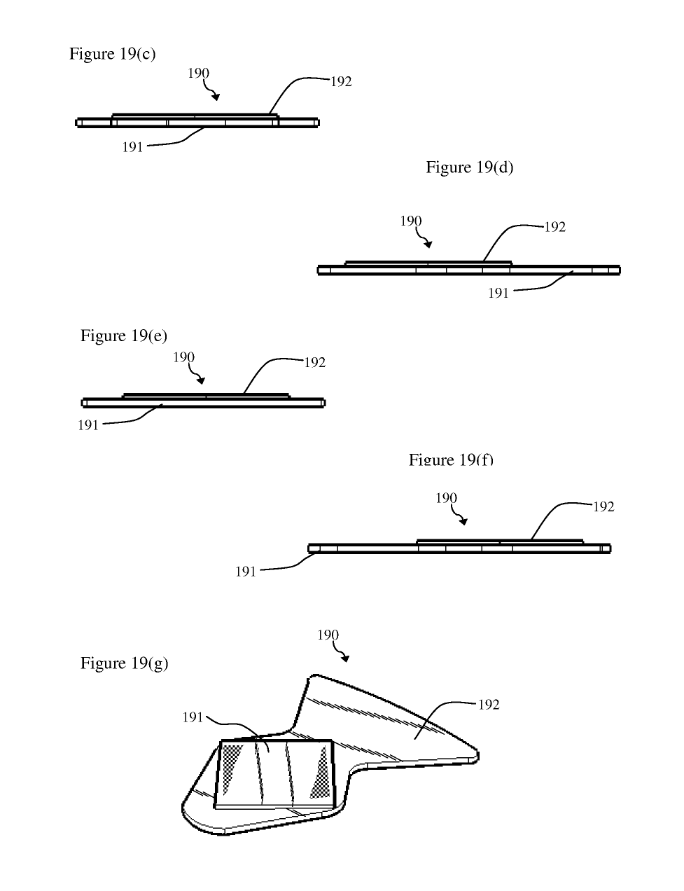

FIGS. 19(a)-19(g) are, respectively, a bottom wound-facing view, a top non-wound facing view, four side views and one perspective view of another exemplary embodiment of a “diamond gauze” bandage 190 according to the present invention. The body portion 191 of the “diamond gauze” bandage 190 has a “knuckle” shape which among other bandaging uses may be particularly useful for bandaging a knuckle as well as a finger tip, or similar appendage. The body portion 191 has a head portion 193 and a tail portion 194. The head portion 193 may be a truncated oval, diamond, almond or similar shape that is possibly asymmetrically shortened on a first side of the head portion 193 where the head portion 193 connects to the tail portion 194. The tail portion 194 flares out with a width that increases as the tail extends lengthwise from the first side of the head portion 193 forming concavities with the head portion 193 along a top edge 195 and bottom edge 196 of the body portion 191 that are preferably smooth curves. The gauze pad 192 is substantially positioned in the head portion 193, and is oriented with respect to the body portion 191 such that the sides of the gauze pad 192 are not parallel to the length tangents 199A, 199C or width tangents 199B, 199D of the body portion 191.

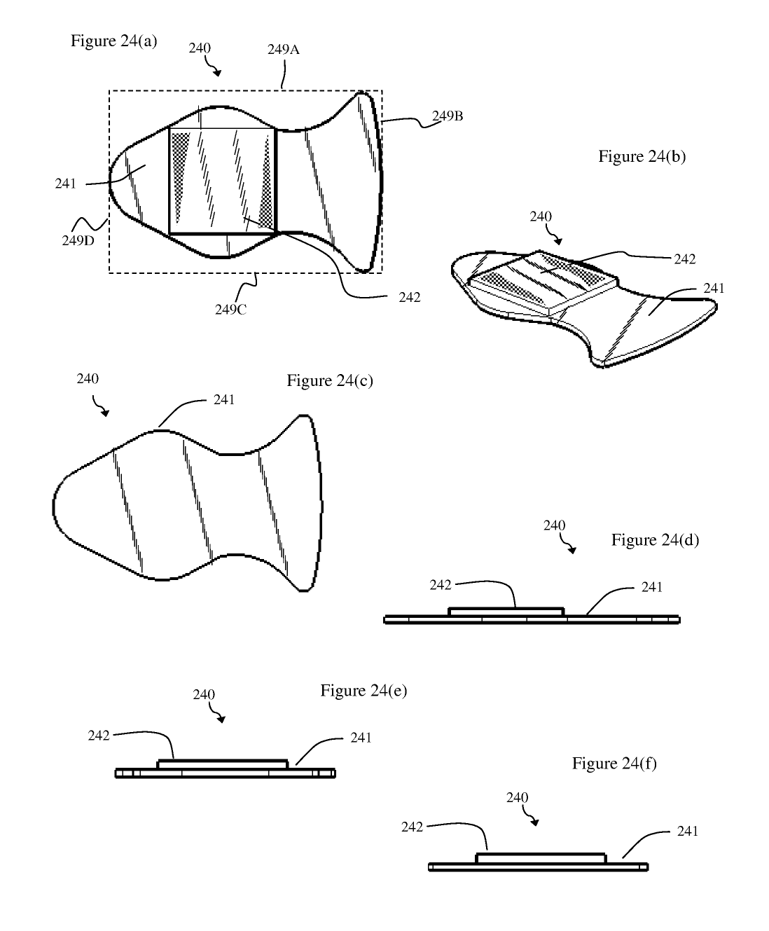

FIGS. 24(a)-24(f) are, respectively, a bottom wound-facing view, a top non-wound facing view, one perspective view, and three side views of another exemplary embodiment of a “diamond gauze” bandage. The body portion 241 of the “diamond gauze” bandage 240 has a “knuckle” shape similar to that of “diamond gauze” bandage 190. The gauze pad 242 is oriented with respect to the “knuckle” shaped body portion 241 such that the sides of the gauze pad 242 are parallel to length tangents 249A, 249C and width tangents 249B, 249D. In all other respects, the “diamond gauze” bandage 240 may be configured in the same manner as “diamond gauze” bandage 190.

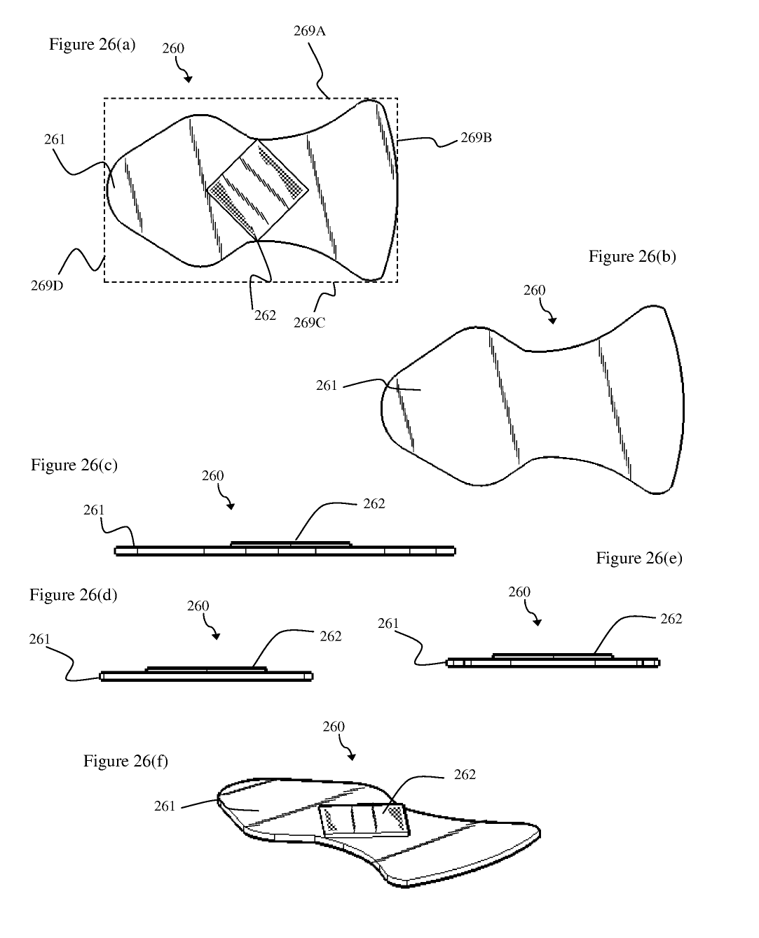

FIGS. 26(a)-26(f) are, respectively, a bottom wound-facing view, a top non-wound facing view, three side views and one perspective view of another exemplary embodiment of a “diamond gauze” bandage. The body portion 261 of the “diamond gauze” bandage 260 has a “knuckle” shape similar to that of “diamond gauze” bandage 190. The gauze pad 262 is positioned so that the center of the gauze pad 262 corresponds to a position along concavities formed by a head portion 263 and a tail portion 264. In all other respects, the “diamond gauze” bandage 260 may be configured in the same manner as “diamond gauze” bandage 190.

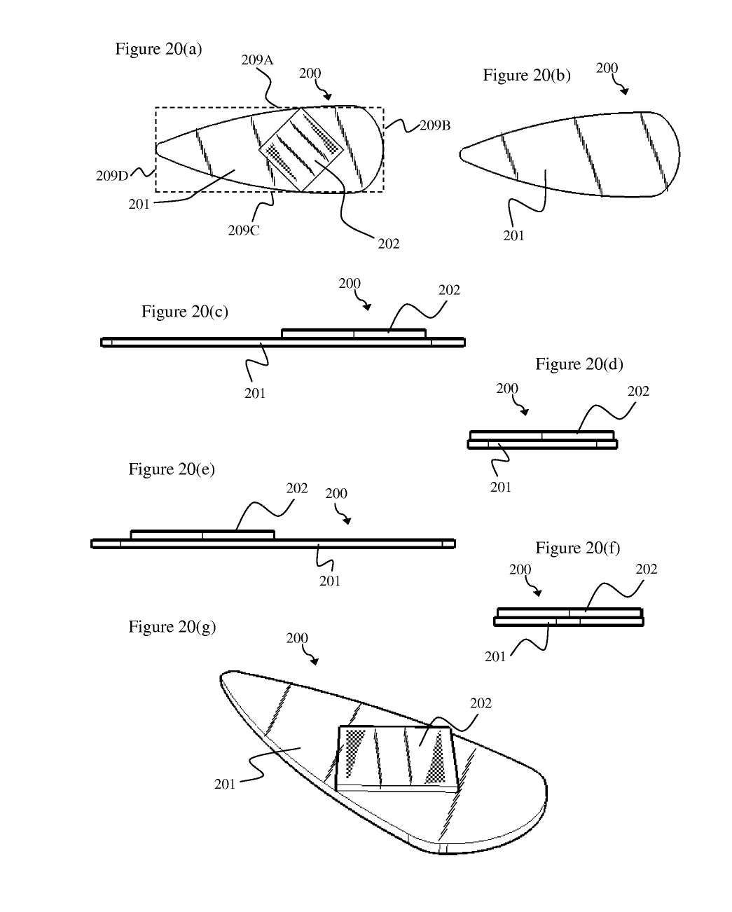

FIGS. 20(a)-20(g) are, respectively, a bottom wound-facing view, a top non-wound facing view, four side views and one perspective view of another exemplary embodiment of a “diamond gauze” bandage 200 according to the present invention. The body portion 201 of the “diamond gauze” bandage 200 has a shape which among other bandaging uses may be particularly useful as a finger wrap. The body portion 201 of the “diamond gauze” bandage 200 may be comprised of self-adherent material, as discussed above by “diamond gauze” bandage 120, so that when it is wrapped around a finger or other appendage, a wound facing side of the bandage that is distal to the gauze pad adheres to the non-wound facing side of the body portion 201 so as to secure the wrap in place. Similarly, all the other “diamond gauze” bandages, particularly when used to wrap around an appendage, may be made with such self-adherent material. The gauze pad 202 is oriented with respect to the body portion 201 such that the sides of the gauze pad 202 are not parallel to length tangents 209A, 209C and width tangents 209B, 209D. The gauze pad 202 is preferably positioned proximate to the width tangent 209B. Alternatively, the “diamond gauze” bandage 200 may be used with the gauze pad 202 not oriented as a diamond shape, but rather in the standard rectangular or square orientation that parallels the length of the bandage, particularly when the “diamond gauze” bandage 200 is configured with self-adherent material or other material for the purpose of resealing the bandage.

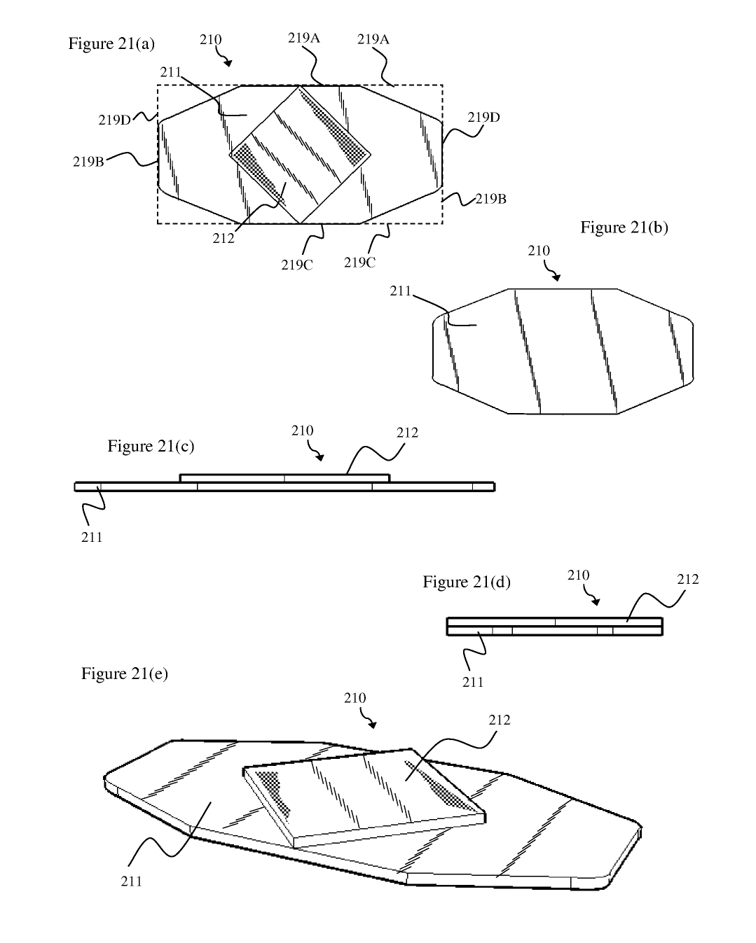

FIGS. 21(a)-21(e) are, respectively, a bottom wound-facing view, a top non-wound facing view, two side views and one perspective view of another exemplary embodiment of a “diamond gauze” bandage 210 according to the present invention. The body portion 211 of the “diamond gauze” bandage 210 is an elongated octagonal shape. The gauze pad 212 is oriented with respect to the body portion 211 such that the sides of the gauze pad 212 are not parallel to length tangents 219A, 219C and width tangents 219B, 219D. The gauze pad 212 may have the same center point as the body portion 211 and/or each of the corners of the gauze pad 212 may be oriented toward a mid-point of one of the length tangents 219A, 219C or width tangents 219B, 219D. Alternatively, the body portion 211 may be an octagonal shape with eight equal sides.

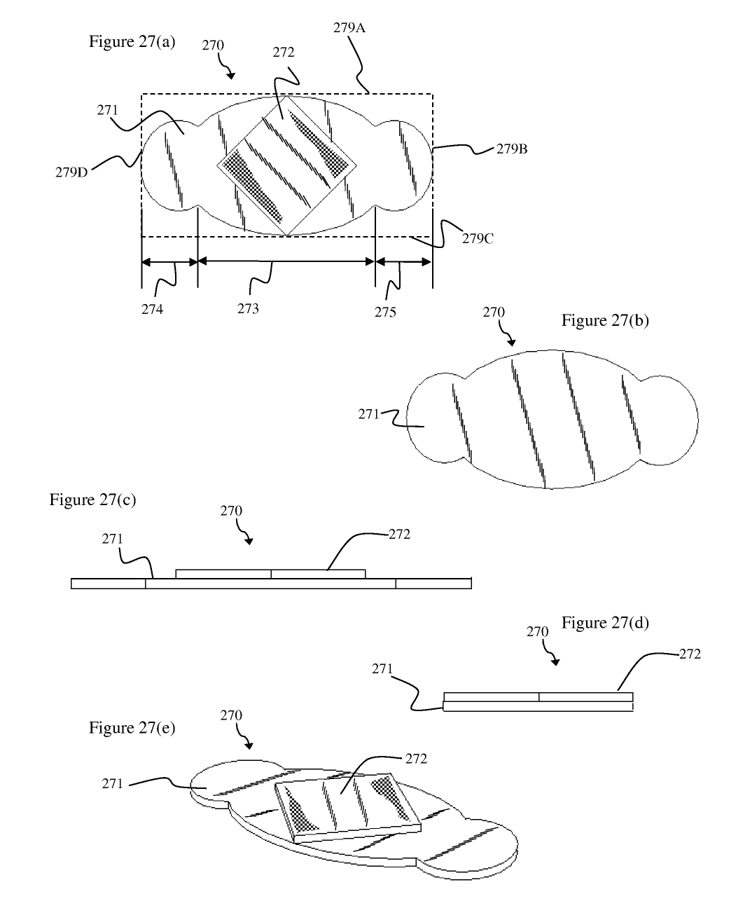

FIGS. 27(a)-27(e) are, respectively, a bottom wound-facing view, a top non-wound facing view, two side views and one perspective view of another exemplary embodiment of a “diamond gauze” bandage 270 according to the present invention. A body portion 271 of the “diamond gauze” bandage 270 has a central almond or oval shaped portion 273 similar to the configuration of the bandages in FIG. 15 or 16 and bulbous side portions 274, 275. The gauze pad 272 is oriented with respect to the body portion 271 such that the sides of the gauze pad 272 are not parallel to length tangents 279A, 279C, and width tangents 279B, 279D. The gauze pad 272 may have the same center point as the body portion 271 and/or each of the corners of the gauze pad 272 may be oriented toward a mid-point of one of the length tangents 279A, 279C or width tangents 279B, 279D. Midpoints of the central portion 273 are preferably positioned to correspond with the midpoints of the length tangents 279A, 279C.

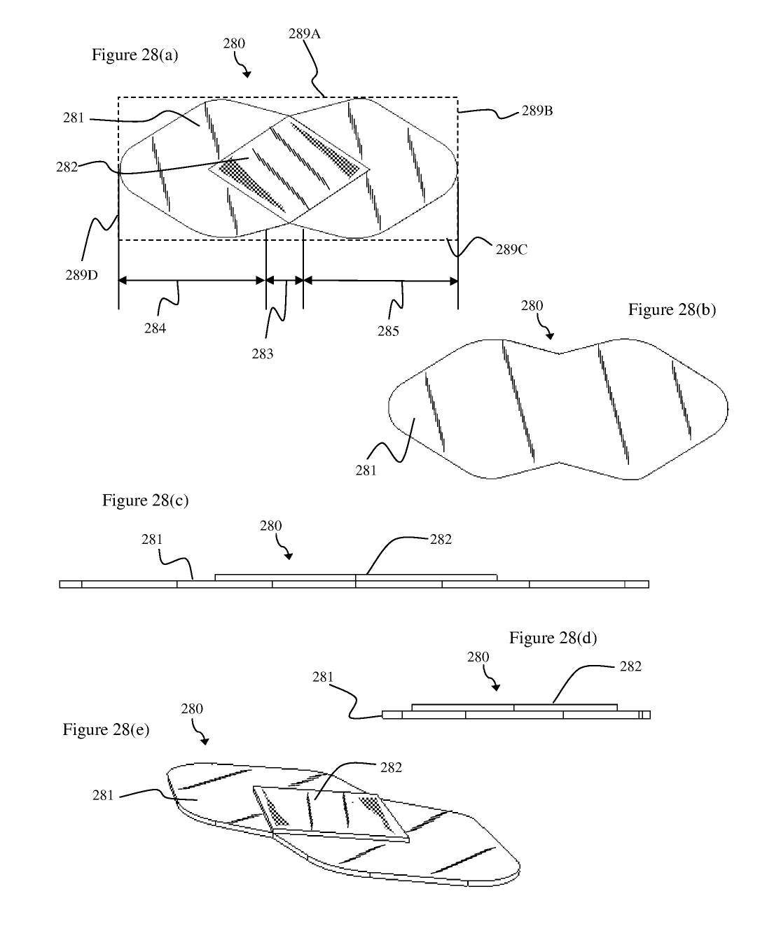

FIGS. 28(a)-28(e) are, respectively, a bottom wound-facing view, a top non-wound facing view, two side views and one perspective view of another exemplary embodiment of a “diamond gauze” bandage 280 according to the present invention. A body portion 281 of the “diamond gauze” bandage 280 has a central narrow portion 283 and side regions 284 and 285 that initially widen, moving along the length from the central narrow portion 283, and then narrowing again in straight or substantially straight lines to rounded ends, where the portions of the side regions 284 and 285 that widen are connected to the portions of the side regions 284 and 285 that narrow by rounded angles. The body portion 281 has a “bowtie” or “hourglass” shape. The gauze pad 282 is oriented with respect to the body portion 281 such that the sides of the gauze pad 282 are not parallel to the length tangents 289A, 289C and width tangents 289B, 289D. The gauze pad 282 may have the same center point as the body portion 281 and/or each of the corners of the gauze pad 282 may be oriented toward a mid-point of one of the length tangents 289A, 289C or width tangents 289B, 289D. The central narrow portion 283 is preferably positioned to correspond with the midpoints of the length tangents 289A, 289C.

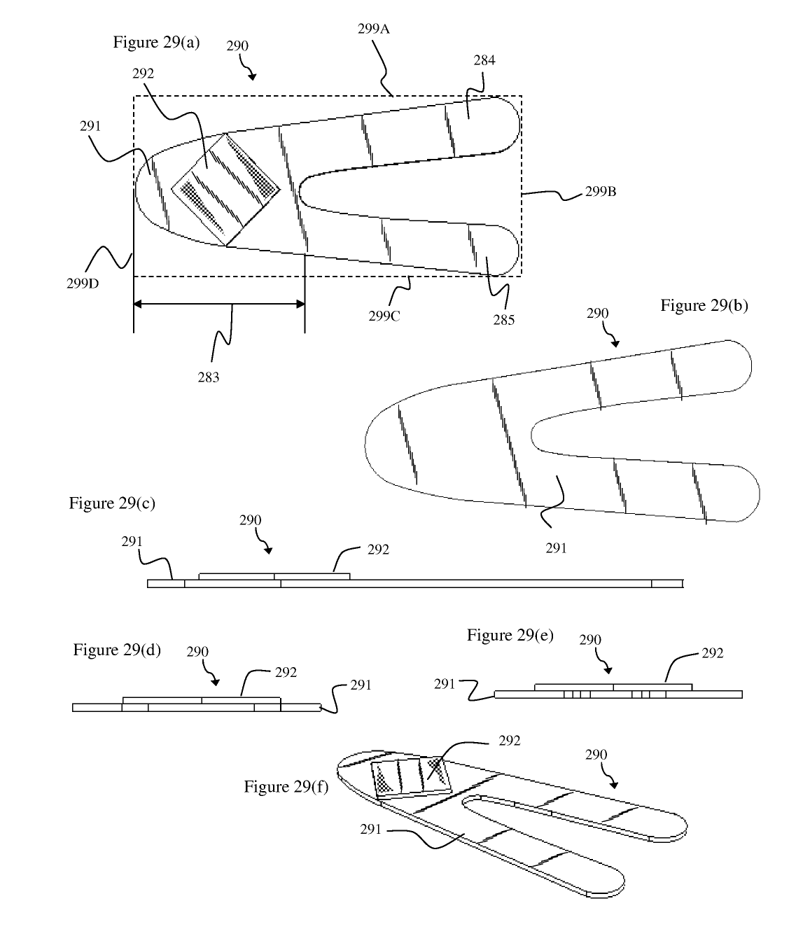

FIGS. 29(a)-29(f) are, respectively, a bottom wound-facing view, a top non-wound facing view, three side views and one perspective view of another exemplary embodiment of a “diamond gauze” bandage 290 according to the present invention. A body portion 291 of the “diamond gauze” bandage 290 has a central portion 293 that lengthwise terminates on a first side with a rounded end, and on a second side has legs 294, 295 that extend from the body portion 291 substantially in the lengthwise direction, the legs 294, 295 terminating with rounded ends distal to the central portion 293. The body portion 291 forms an “arch” shape defined by two parallel arcs or parabolas that are connected at each of their respective proximate ends distal to the central portion 293 by a third and fourth curve forming convexities on the terminating ends of the legs 294, 295. Alternatively, the body portion 291 may be formed with straight or wavy sides rather than the parabolas and may include squared off ends rather than the convexities on the ends of the legs 294, 295 and the central portion 293. The legs 294, 295 are spaced apart from each other at the central portion 293 and preferably extend out at diverging angles from the central portion 293. The diverging angles should preferably allow the legs 294, 295 to extend around a limb to which the “diamond gauze” bandage 290 is being applied and to have the bottom wound-facing side of the legs 294, 295 slightly overlap on top of the top non-wound facing side of the central portion 295. Thus, the body portion 291 has a shape which among other bandaging uses may be particularly useful as a finger wrap. The gauze pad 292 is oriented with respect to the body portion 291 such that the sides of the gauze pad 292 are not parallel to length tangents 299A, 299C and width tangents 299B, 299D. The gauze pad 292 may have the same center point as the central body portion 293 and/or corners of the gauze pad 292 may be oriented toward a mid-point of one of the width tangents 299B, 299D.

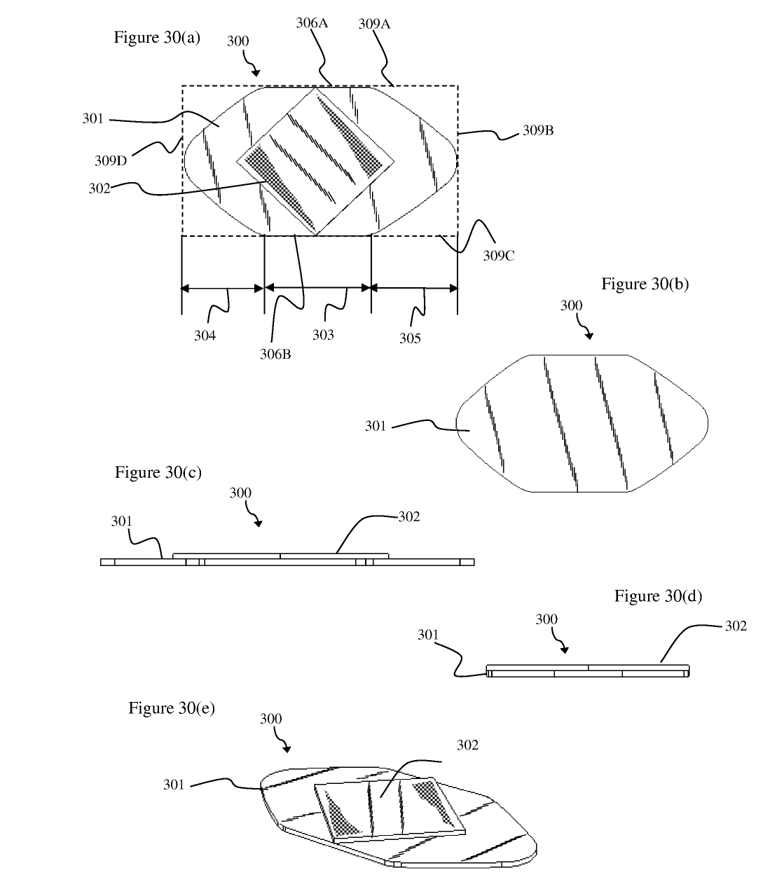

FIGS. 30(a)-30(e) are, respectively, a bottom wound-facing view, a top non-wound facing view, two side views and one perspective view of another exemplary embodiment of a “diamond gauze” bandage 300 according to the present invention. A body portion 301 of the “diamond gauze” bandage 300 has a central portion 303 that has flattened top and bottom edges 306A, 306B, resulting in a uniform or substantially uniform width for the central portion 303. The body portion 301 of the “diamond gauze” bandage 300 also has side regions 304 and 305 that, moving along the length from the central portion 303, narrow in straight or substantially straight lines to rounded ends, where the central portion 303 transitions to the side regions 304 and 305 by rounded angles. The gauze pad 302 is oriented with respect to the body portion 301 such that the sides of the gauze pad 302 are not parallel to length tangents 309A, 309C and width tangents 309B, 309D. The gauze pad 302 may have the same center point as the body portion 301 and/or each of the corners of the gauze pad 302 may be oriented toward a mid-point of one of the length tangents 309A, 309C or width tangents 309B, 309D. Midpoints of the central portion 303 are preferably positioned to correspond with the midpoints of the length tangents 309A, 309C.

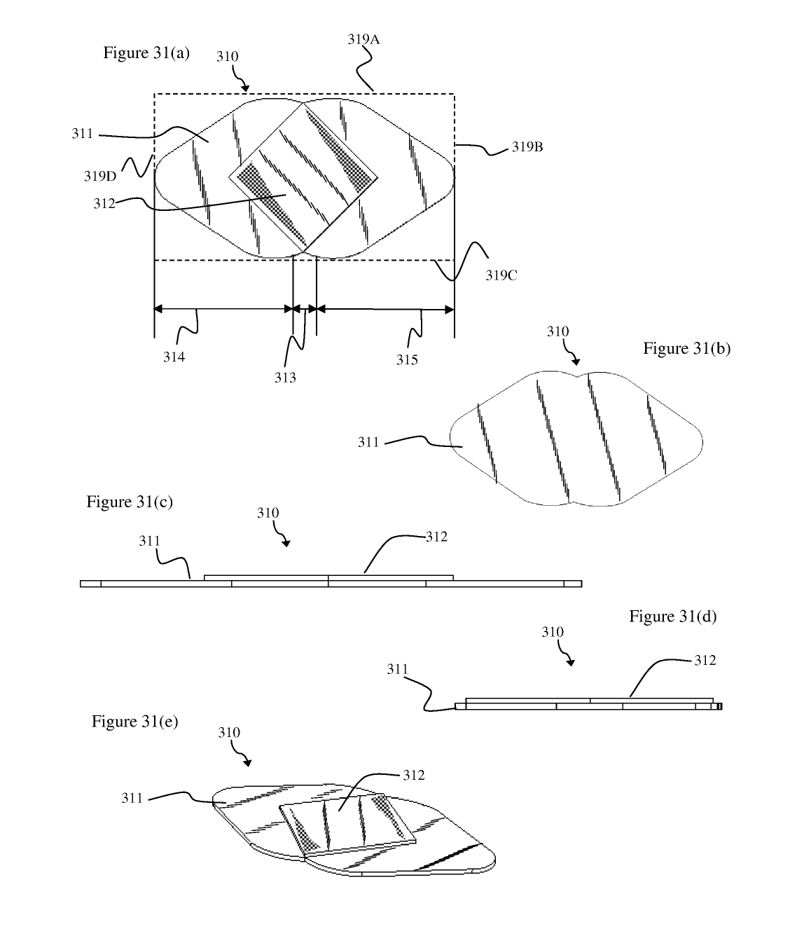

FIGS. 31(a)-31(e) are, respectively, a bottom wound-facing view, a top non-wound facing view, two side views and one perspective view of another exemplary embodiment of a “diamond gauze” bandage 310 according to the present invention. A body portion 311 of the “diamond gauze” bandage 310 has a central narrow portion 313 and side regions 314 and 315 that initially widen, moving along the length from the central narrow portion 313, and then narrowing again in straight or substantially lines to rounded ends. The “diamond gauze” bandage 310 has a body portion that has a “bowtie” or “hourglass” shape. The gauze pad 312 is oriented with respect to the body portion 311 such that the sides of the gauze pad 312 are not parallel to length tangents 319A, 319C and width tangents 319B, 319D. The gauze pad 312 may have the same center point as the body portion 311 and/or each of the corners of the gauze pad 312 may be oriented toward a mid-point of one of the length tangents 319A, 319C or width tangents 319B, 319D. The central narrow portion 313 is preferably positioned to correspond with the midpoints of the length tangents 319A, 319C.

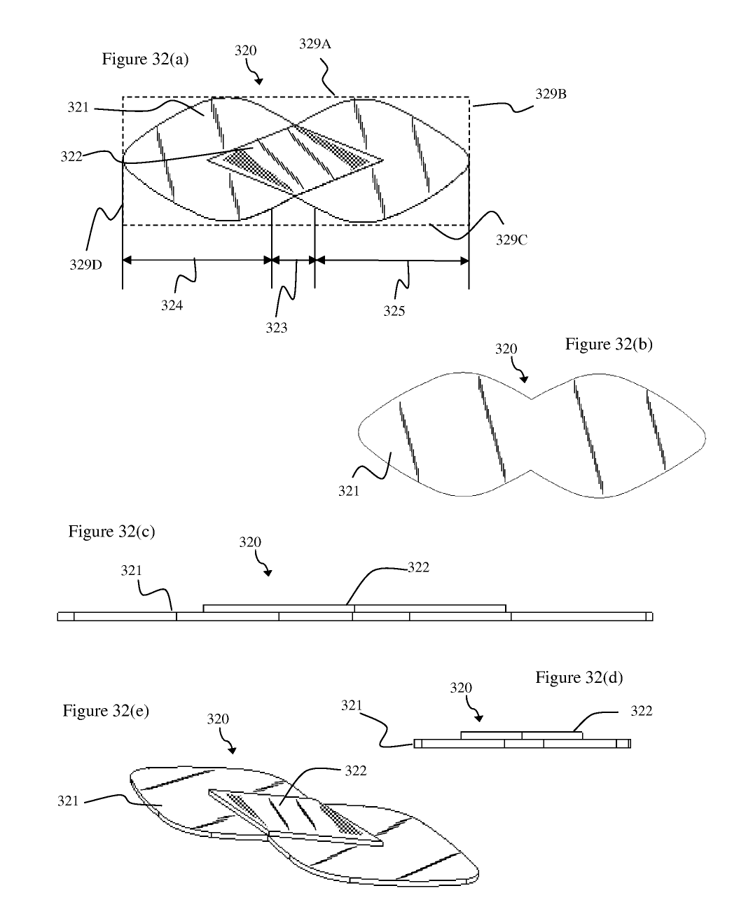

FIGS. 32(a)-32(e) are, respectively, a bottom wound-facing view, a top non-wound facing view, two side views and one perspective view of another exemplary embodiment of a “diamond gauze” bandage 320 according to the present invention. A body portion 321 of the “diamond gauze” bandage 320 has a central narrow portion 323 and side regions 324 and 325 that initially widen, moving along the length from the central narrow portion 323, and then narrowing again in straight or substantially lines to rounded ends. The “diamond gauze” bandage 320 has a body portion that has a “bowtie” or “hourglass” shape. The gauze pad 322 is oriented with respect to the body portion 321 such that the sides of the gauze pad 322 are not parallel to length tangents 329A, 329C and width tangents 329B, 329D. The gauze pad 322 may have the same center point as the body portion 321 and/or each of the corners of the gauze pad 322 may be oriented toward a mid-point of one of the length tangents 329A, 329C or width tangents 329B, 329D. The central narrow portion 323 is preferably positioned to correspond with the midpoints of the length tangents 329A, 329C.

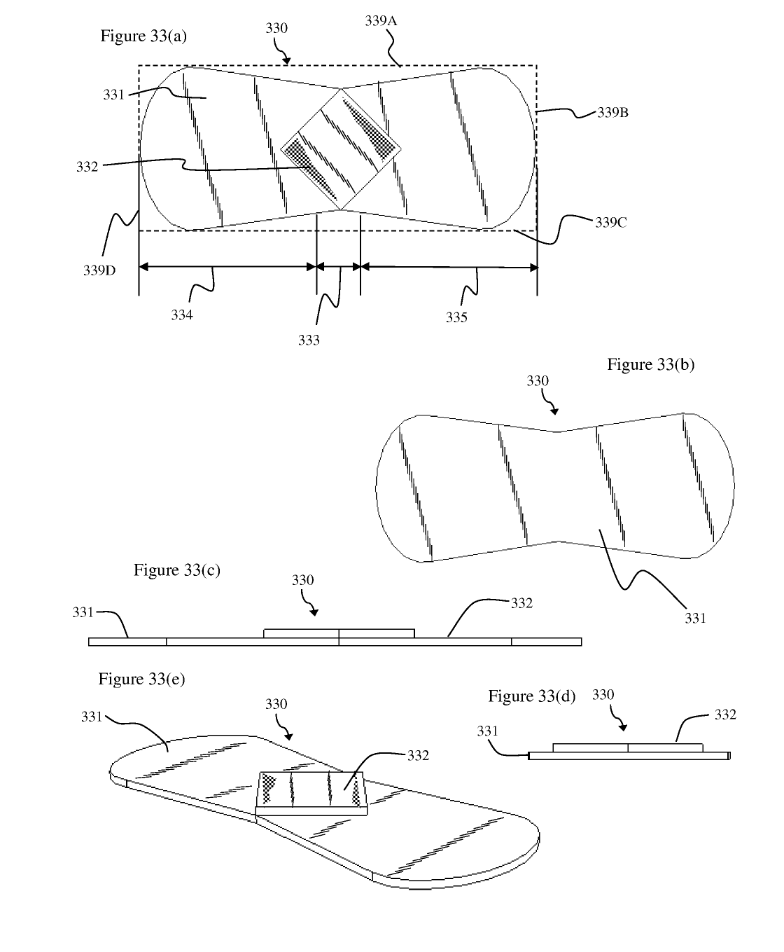

FIGS. 33(a)-33(e) are, respectively, a bottom wound-facing view, a top non-wound facing view, two side views and one perspective view of another exemplary embodiment of a “diamond gauze” bandage 330 according to the present invention. A body portion 331 of the “diamond gauze” bandage 330 has a central narrow portion 333 and side regions 334 and 335 that widen in straight or substantially straight lines to rounded ends. The “diamond gauze” bandage 330 has a body portion that has a “bowtie” or “hourglass” shape. The gauze pad 332 is oriented with respect to the body portion 331 such that the sides of the gauze pad 332 are not parallel to length tangents 339A, 339C and width tangents 339B, 339D. The gauze pad 332 may have the same center point as the body portion 331 and/or each of the corners of the gauze pad 332 may be oriented toward a mid-point of one of the length tangents 339A, 339C or width tangents 339B, 339D. The central narrow portion 333 is preferably positioned to correspond with the midpoints of the length tangents 339A, 339C.

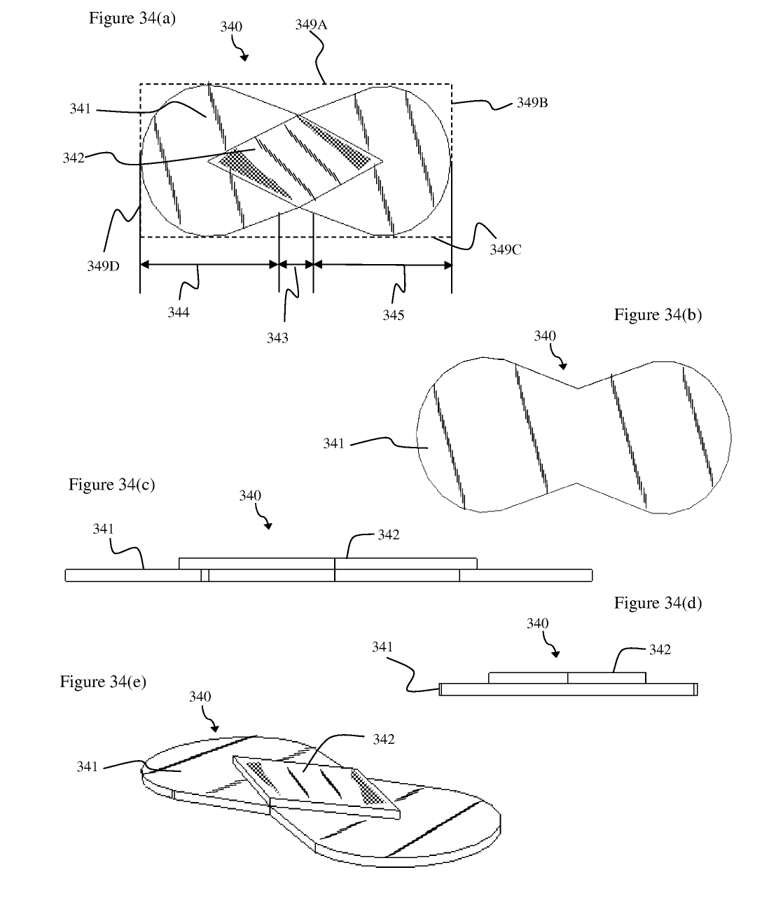

FIGS. 34(a)-34(e) are, respectively, a bottom wound-facing view, a top non-wound facing view, two side views and one perspective view of another exemplary embodiment of a “diamond gauze” bandage 340 according to the present invention. A body portion 341 of the “diamond gauze” bandage 340 has a central narrow portion 343 and side regions 344 and 345 that widen in straight or substantially straight lines to rounded ends. The “diamond gauze” bandage 340 has a body portion that has a “bowtie” or “hourglass” shape. The gauze pad 342 is oriented with respect to the body portion 341 such that the sides of the gauze pad 342 are not parallel to length tangents 349A, 349C and width tangents 349B, 349D. The gauze pad 342 may have the same center point as the body portion 341 and/or each of the corners of the gauze pad 342 may be oriented toward a mid-point of one of the length tangents 349A, 349C or width tangents 349B, 349D. The central narrow portion 343 is preferably positioned to correspond with the midpoints of the length tangents 349A, 349C.

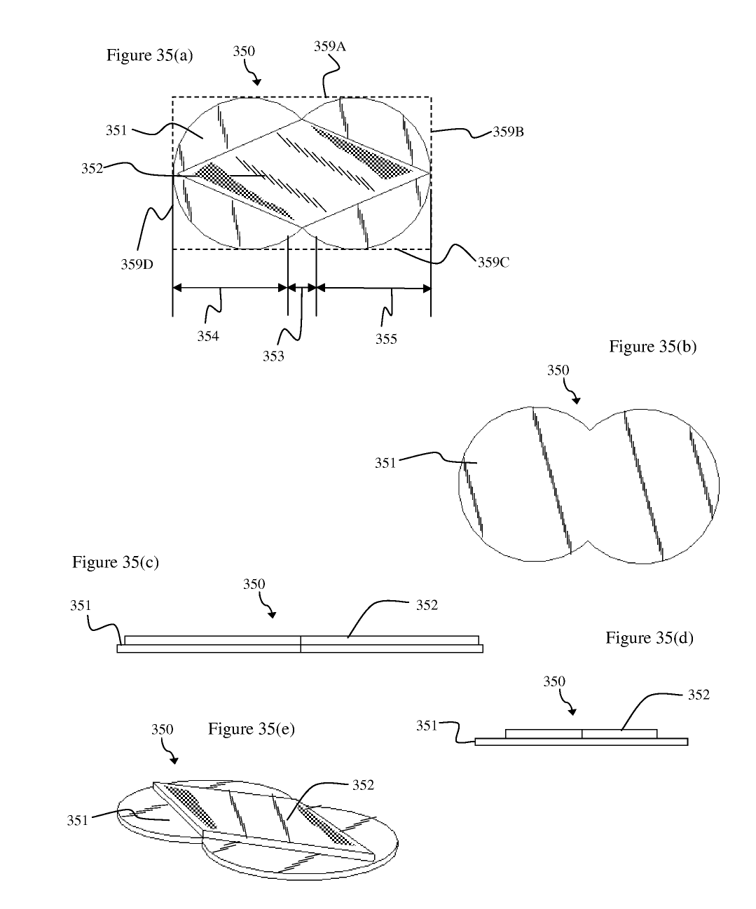

FIGS. 35(a)-35(e) are, respectively, a bottom wound-facing view, a top non-wound facing view, two side views and one perspective view of another exemplary embodiment of a “diamond gauze” bandage 350 according to the present invention. A body portion 351 of the “diamond gauze” bandage 350 has a central narrow portion 353 and side regions 354 and 355 that are formed by circular or elliptical arcs. The “diamond gauze” bandage 350 has a body portion that has a “bowtie” or “hourglass” shape. The gauze pad 352 is oriented with respect to the body portion 351 such that the sides of the gauze pad 352 are not parallel to length tangents 359A, 359C and width tangents 359B, 359D. The gauze pad 352 may have the same center point as the body portion 351 and/or each of the corners of the gauze pad 352 may be oriented toward a mid-point of one of the length tangents 359A, 359C or width tangents 359B, 359D. The central narrow portion 353 is preferably positioned to correspond with the midpoints of the length tangents 359A, 359C.

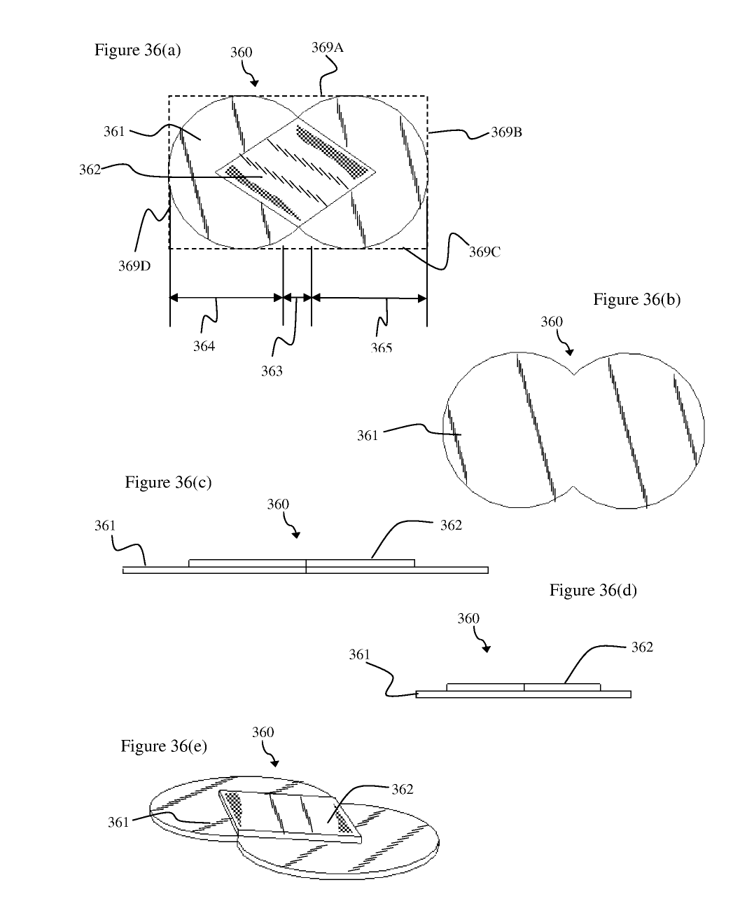

FIGS. 36(a)-36(e) are, respectively, a bottom wound-facing view, a top non-wound facing view, two side views and one perspective view of another exemplary embodiment of a “diamond gauze” bandage 360 according to the present invention. A body portion 361 of the “diamond gauze” bandage 360 has a central narrow portion 363 and side regions 364 and 365 that are formed by circular or elliptical arcs. The “diamond gauze” bandage 360 has a body portion that has a “bowtie” or “hourglass” shape. The gauze pad 362 is oriented with respect to the body portion 361 such that the sides of the gauze pad 362 are not parallel to length tangents 369A, 369C and width tangents 369B, 369D. The gauze pad 362 may have the same center point as the body portion 361 and/or each of the corners of the gauze pad 362 may be oriented toward a mid-point of one of the length tangents 369A, 369C or width tangents 369B, 369D. The central narrow portion 363 is preferably positioned to correspond with the midpoints of the length tangents 369A, 369C.

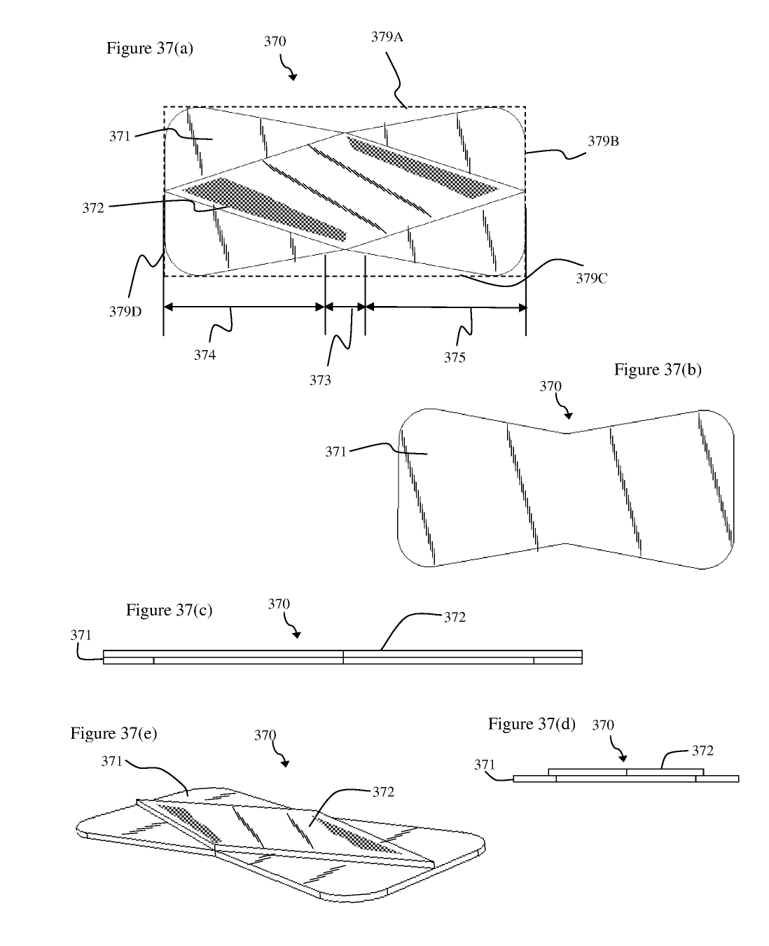

FIGS. 37(a)-37(e) are, respectively, a bottom wound-facing view, a top non-wound facing view, two side views and one perspective view of another exemplary embodiment of a “diamond gauze” bandage 370 according to the present invention. A body portion 371 of the “diamond gauze” bandage 370 has a central narrow portion 373 and side regions 374 and 375 that widen in straight or substantially straight lines to straight or substantially straight ends, connected by rounded corners. The “diamond gauze” bandage 370 has a body portion that has a “bowtie” or “hourglass” shape with flattened ends. The gauze pad 372 is oriented with respect to the body portion 371 such that the sides of the gauze pad 372 are not parallel to length tangents 379A, 379C and width tangents 379B, 379D. The gauze pad 372 may have the same center point as the body portion 371 and/or each of the corners of the gauze pad 372 may be oriented toward a mid-point of one of the length tangents 379A, 379C or width tangents 379B, 379D. The central narrow portion 373 is preferably positioned to correspond with the midpoints of the length tangents 379A, 379C.

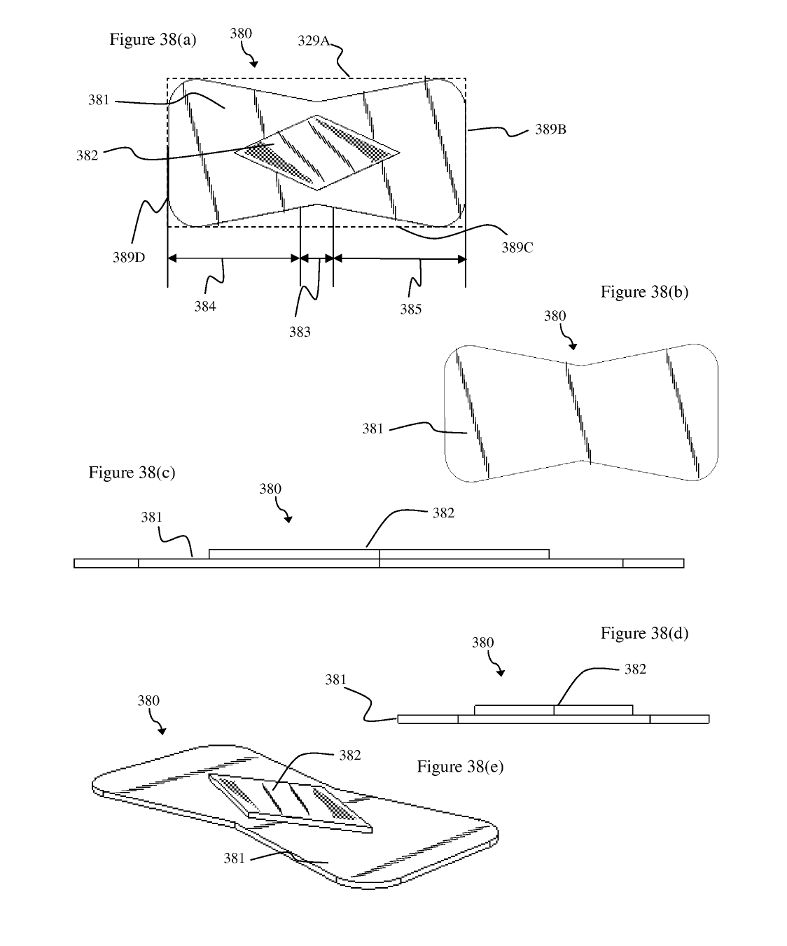

FIGS. 38(a)-38(e) are, respectively, a bottom wound-facing view, a top non-wound facing view, two side views and one perspective view of another exemplary embodiment of a “diamond gauze” bandage 380 according to the present invention. A body portion 381 of the “diamond gauze” bandage 380 has a central narrow portion 383 and side regions 384 and 385 that widen in straight or substantially straight lines to straight or substantially straight ends, connected by rounded corners. The “diamond gauze” bandage 380 has a body portion that has a “bowtie” or “hourglass” shape with flattened ends. The gauze pad 382 is oriented with respect to the body portion 381 such that the sides of the gauze pad 382 are not parallel to length tangents 389A, 389C and width tangents 389B, 389D. The gauze pad 382 may have the same center point as the body portion 381 and/or each of the corners of the gauze pad 382 may be oriented toward a mid-point of one of the length tangents 389A, 389C or width tangents 389B, 389D. The central narrow portion 383 is preferably positioned to correspond with the midpoints of the length tangents 389A, 389C.

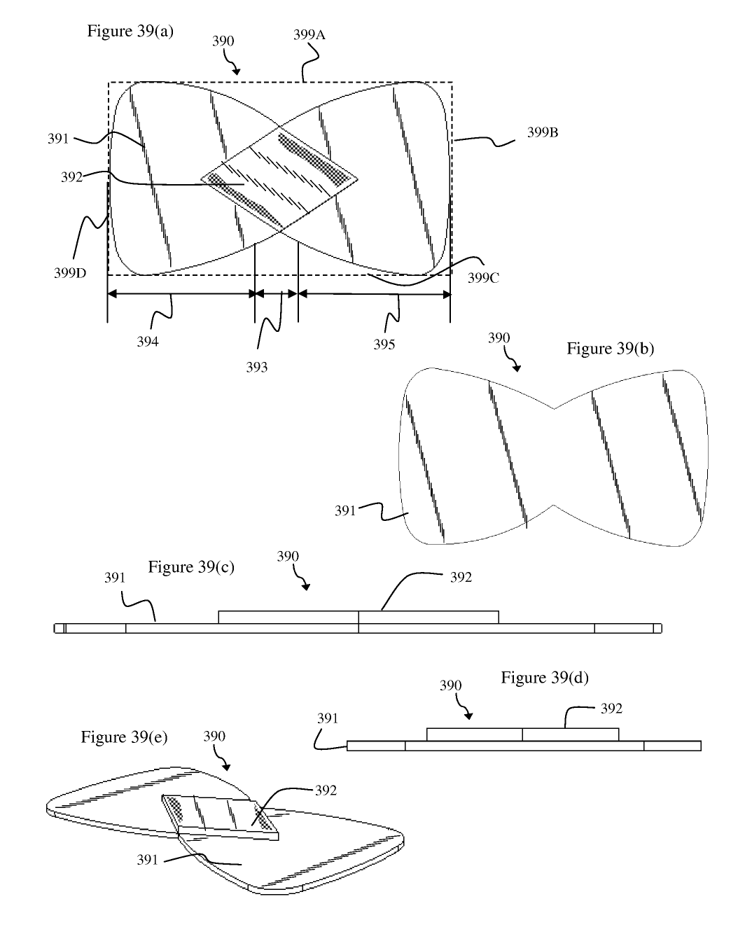

FIGS. 39(a)-39(e) are, respectively, a bottom wound-facing view, a top non-wound facing view, two side views and one perspective view of another exemplary embodiment of a “diamond gauze” bandage 390 according to the present invention. A body portion 391 of the “diamond gauze” bandage 390 has a central narrow portion 393 and side regions 394 and 395 that widen in curved lines to straight or substantially straight ends, connected by rounded corners. The “diamond gauze” bandage 390 has a body portion that has a “bowtie” or “hourglass” shape with flattened ends. The gauze pad 392 is oriented with respect to the body portion 391 such that the sides of the gauze pad 392 are not parallel to length tangents 399A, 399C and width tangents 399B, 399D. The gauze pad 392 may have the same center point as the body portion 391 and/or each of the corners of the gauze pad 392 may be oriented toward a mid-point of one of the length tangents 399A, 399C or width tangents 399B, 399D. The central narrow portion 393 is preferably positioned to correspond with the midpoints of the length tangents 399A, 399C.

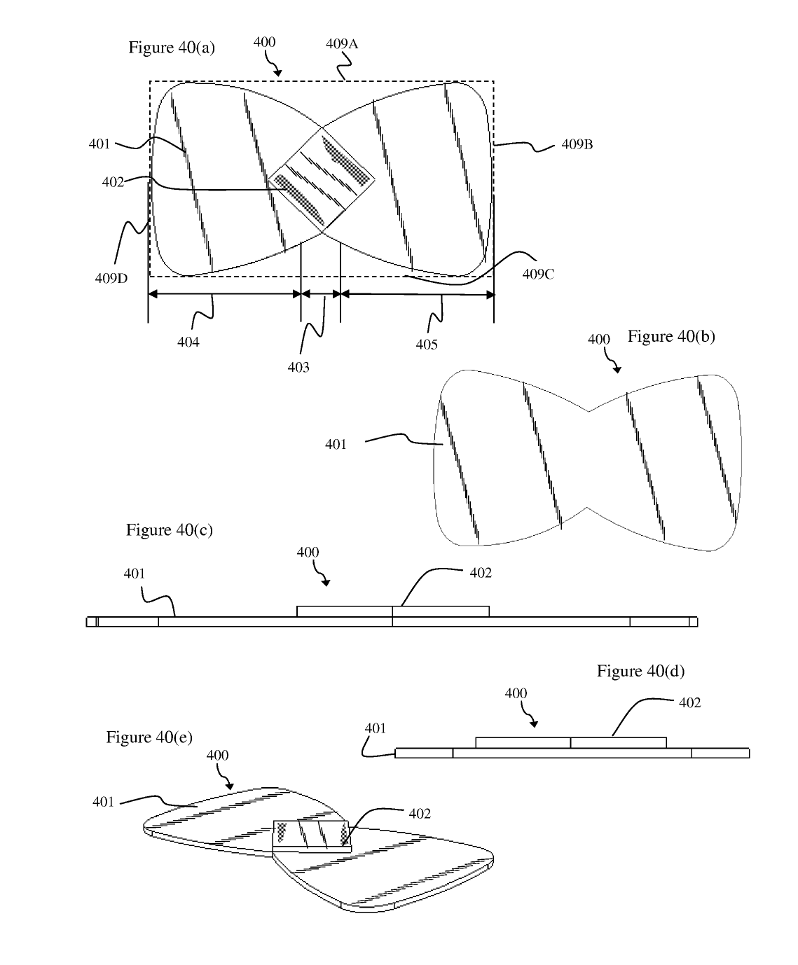

FIGS. 40(a)-40(e) are, respectively, a bottom wound-facing view, a top non-wound facing view, two side views and one perspective view of another exemplary embodiment of a “diamond gauze” bandage 400 according to the present invention. A body portion 401 of the “diamond gauze” bandage 400 has a central narrow portion 403 and side regions 404 and 405 that widen in curved lines to straight or substantially straight ends, connected by rounded corners. The “diamond gauze” bandage 400 has a body portion that has a “bowtie” or “hourglass” shape with flattened ends. The gauze pad 402 is oriented with respect to the body portion 401 such that the sides of the gauze pad 402 are not parallel to length tangents 409A, 409C and width tangents 409B, 409D. The gauze pad 402 may have the same center point as the body portion 401 and/or each of the corners of the gauze pad 402 may be oriented toward a mid-point of one of the length tangents 409A, 409C or width tangents 409B, 409D. The central narrow portion 403 is preferably positioned to correspond with the midpoints of the length tangents 409A, 409C.

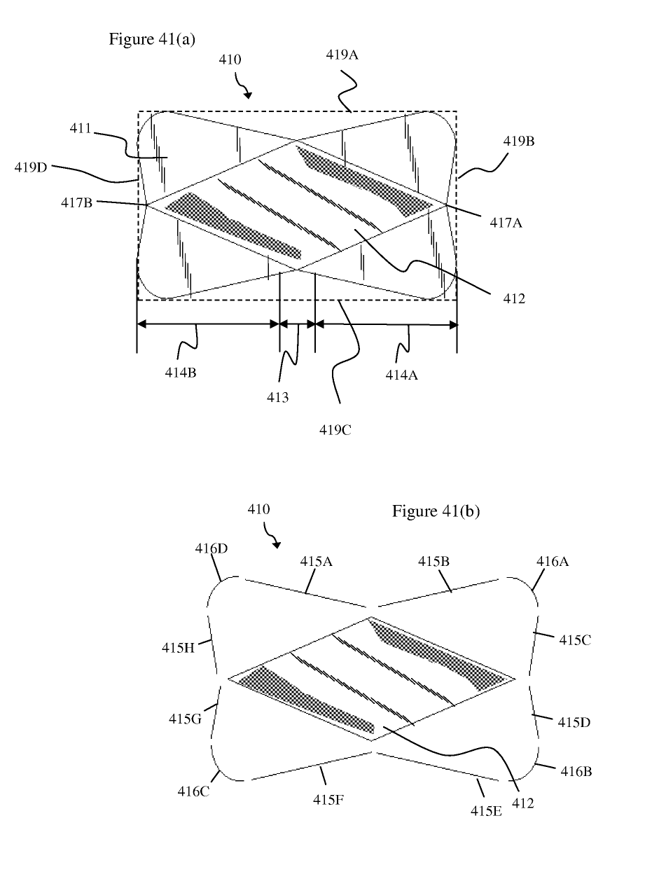

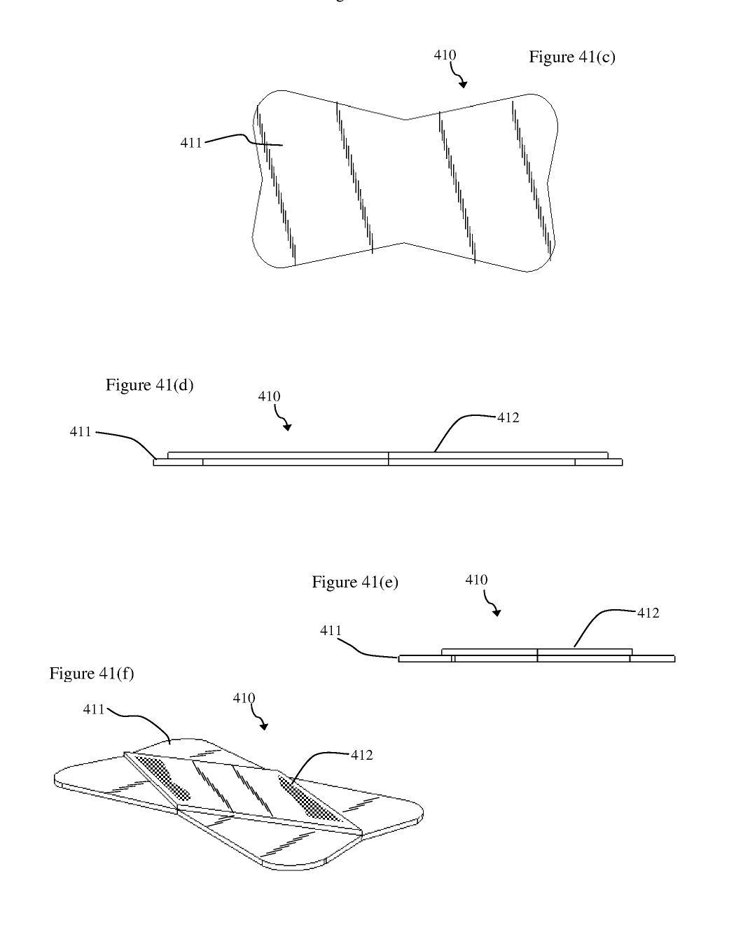

FIGS. 41(a)-41(f) are, respectively, a bottom wound-facing view, an exploded bottom wound-facing view, a top non-wound facing view, two side views and one perspective view of another exemplary embodiment of a “diamond gauze” bandage 410 according to the present invention. A body portion 411 of the “diamond gauze” bandage 410 has a central narrow portion 413 and side regions 414A, 414B that widen with straight or substantially straight side lines 415A, 415B, 415E, 415F as the side regions extend to rounded corners 416A, 416B, 416C, 416D. The rounded corners 416A, 416B, 416C, 416D transition to straight or substantially straight end lines 415C, 415D, 415G, 415H that are oriented so as to taper or decrease the length of the body portion 411 as the substantially straight end lines 415C, 415D, 415G, 415H extend toward inflection points 417A, 417B. The inflection points 417A, 417B are preferably at midpoints between corresponding rounded corners 416A, 416B, 416C, 416D. The side lines 415A, 415B, 415E, 415F are preferably equal in length, and longer than the end lines 415H, 415C, 415D, 415G, respectively, which are also preferably equal in length. The “diamond gauze” bandage 410 has a body portion that has a “bowtie” or “hourglass” shape. The gauze pad 412 is oriented with respect to the body portion 411 such that the sides of the gauze pad 412 are not parallel to length tangents 419A, 419C and width tangents 419B, 419D. The gauze pad 412 may have the same center point as the body portion 411 and/or each of the corners of the gauze pad 412 may be oriented toward a mid-point of one of the length tangents 419A, 419C or width tangents 419B, 419D. The central narrow portion 413 is preferably positioned to correspond with the midpoints of the length tangents 419A, 419C.

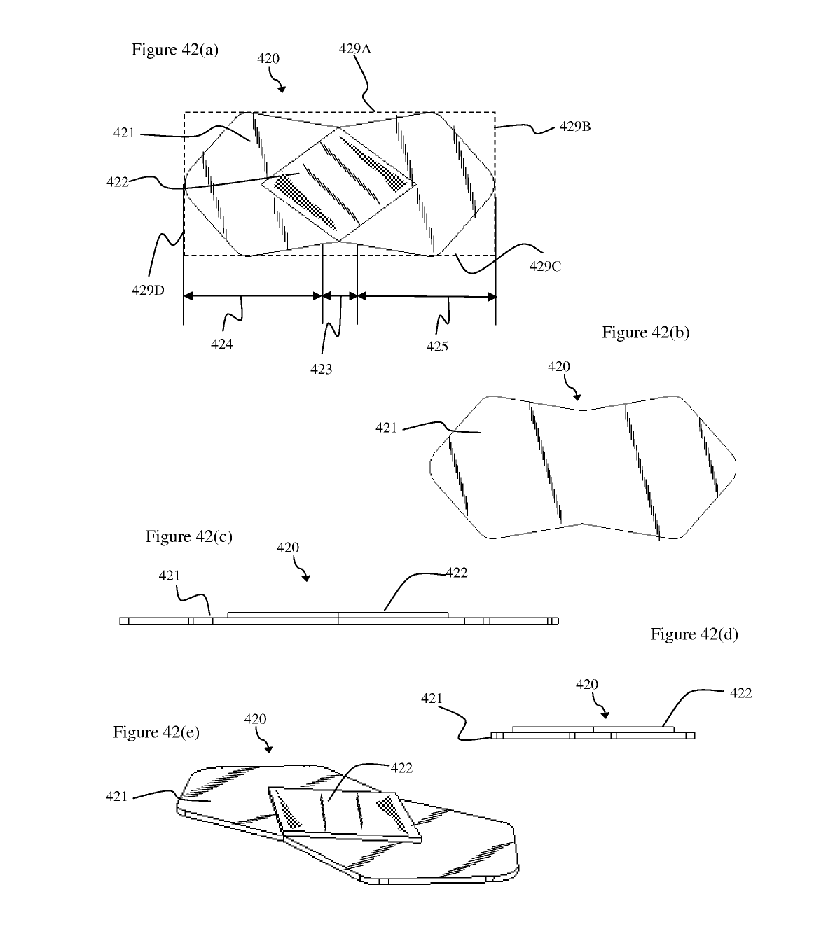

FIGS. 42(a)-42(e) are, respectively, a bottom wound-facing view, a top non-wound facing view, two side views and one perspective view of another exemplary embodiment of a “diamond gauze” bandage 420 according to the present invention. A body portion 421 of the “diamond gauze” bandage 420 has a central narrow portion 423 and side regions 424 and 425 that initially widen, moving along the length from the central narrow portion 423, and then narrowing again in straight or substantially straight lines to rounded ends, where the portions of the side regions 424 and 425 that widen are connected to the portions of the side regions 424 and 425 that narrow by rounded angles. The body portion 421 has a “bowtie” or “hourglass” shape. The gauze pad 422 is oriented with respect to the body portion 421 such that the sides of the gauze pad 422 are not parallel to length tangents 429A, 429C and width tangents 429B, 429D. The gauze pad 422 may have the same center point as the body portion 421 and/or each of the corners of the gauze pad 422 may be oriented toward a mid-point of one of the length tangents 429A, 429C or width tangents 429B, 429D. The central narrow portion 423 is preferably positioned to correspond with the midpoints of the length tangents 429A, 429C.

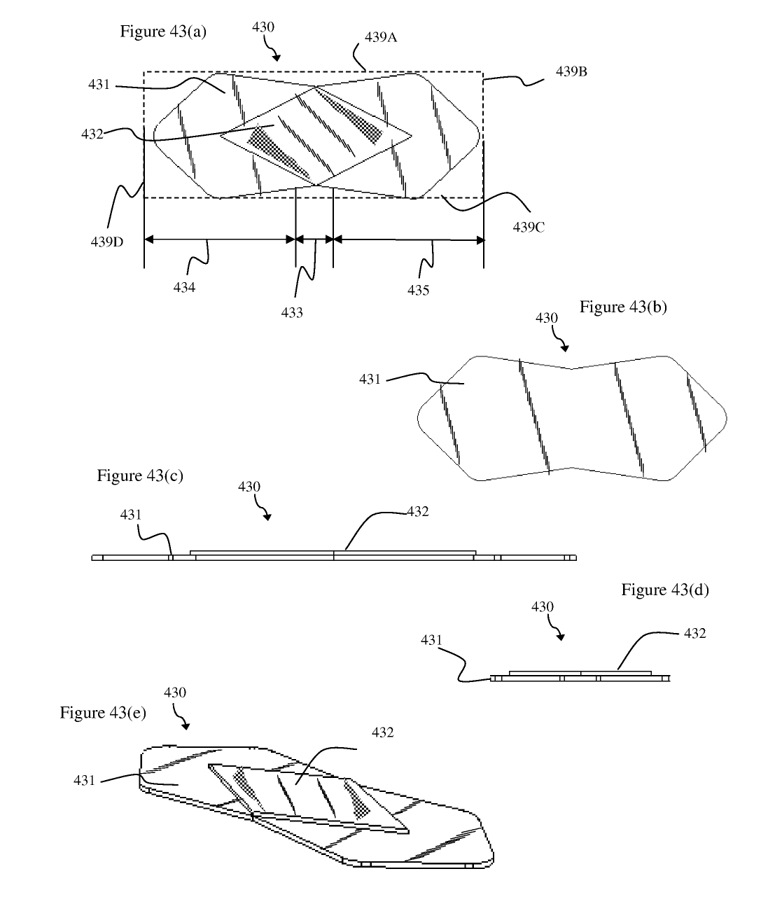

FIGS. 43(a)-43(e) are, respectively, a bottom wound-facing view, a top non-wound facing view, two side views and one perspective view of another exemplary embodiment of a “diamond gauze” bandage 430 according to the present invention. A body portion 431 of the “diamond gauze” bandage 430 has a central narrow portion 433 and side regions 434 and 435 that initially widen, moving along the length from the central narrow portion 433, and then narrowing again in straight or substantially straight lines to rounded ends, where the portions of the side regions 434 and 435 that widen are connected to the portions of the side regions 434 and 435 that narrow by rounded angles. The body portion 431 has a “bowtie” or “hourglass” shape. The gauze pad 432 is oriented with respect to the body portion 431 such that the sides of the gauze pad 432 are not parallel to length tangents 439A, 439C and width tangents 439B, 439D. The gauze pad 432 may have the same center point as the body portion 431 and/or each of the corners of the gauze pad 432 may be oriented toward a mid-point of one of the length tangents 439A, 439C or width tangents 439B, 439D. The central narrow portion 433 is preferably positioned to correspond with the midpoints of the length tangents 439A, 439C.

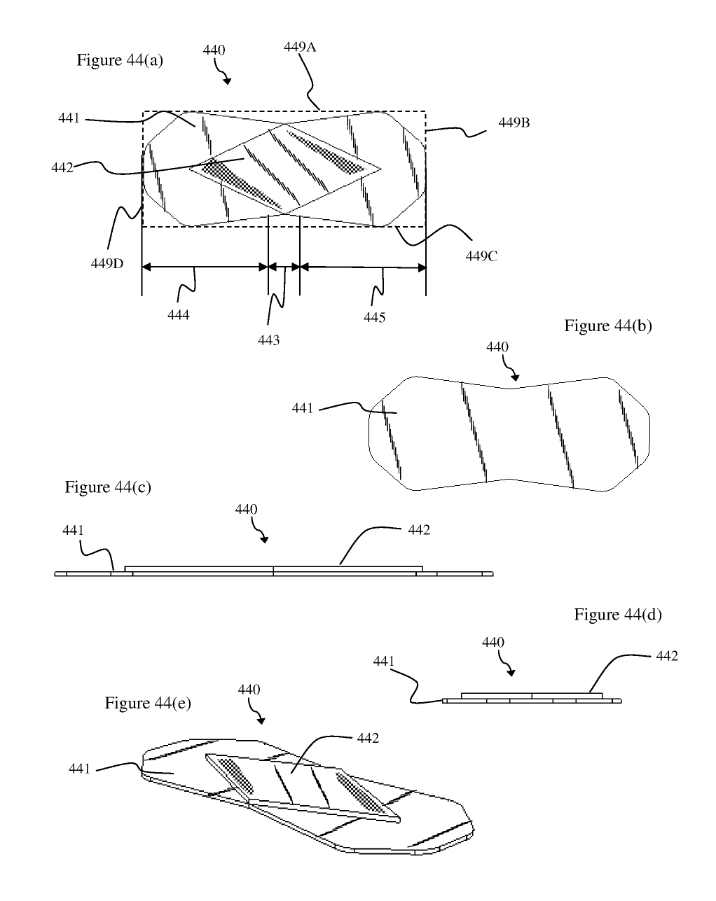

FIGS. 44(a)-44(e) are, respectively, a bottom wound-facing view, a top non-wound facing view, two side views and one perspective view of another exemplary embodiment of a “diamond gauze” bandage 440 according to the present invention. A body portion 441 of the “diamond gauze” bandage 440 has a central narrow portion 443 and side regions 444 and 445 that initially widen, moving along the length from the central narrow portion 443, and then narrowing again in straight or substantially straight lines to straight or substantially straight ends connected by first rounded angles, where the portions of the side regions 444 and 445 that widen are connected to the portions of the side regions 444 and 445 that narrow by second rounded angles. The body portion 441 has a “bowtie” or “hourglass” shape with flattened ends. The gauze pad 442 is oriented with respect to the body portion 441 such that the sides of the gauze pad 442 are not parallel to length tangents 449A, 449C and width tangents 449B, 449D. The gauze pad 442 may have the same center point as the body portion 441 and/or each of the corners of the gauze pad 442 may be oriented toward a mid-point of one of the length tangents 449A, 449C or width tangents 449B, 449D. The central narrow portion 443 is preferably positioned to correspond with the midpoints of the length tangents 449A, 449C.

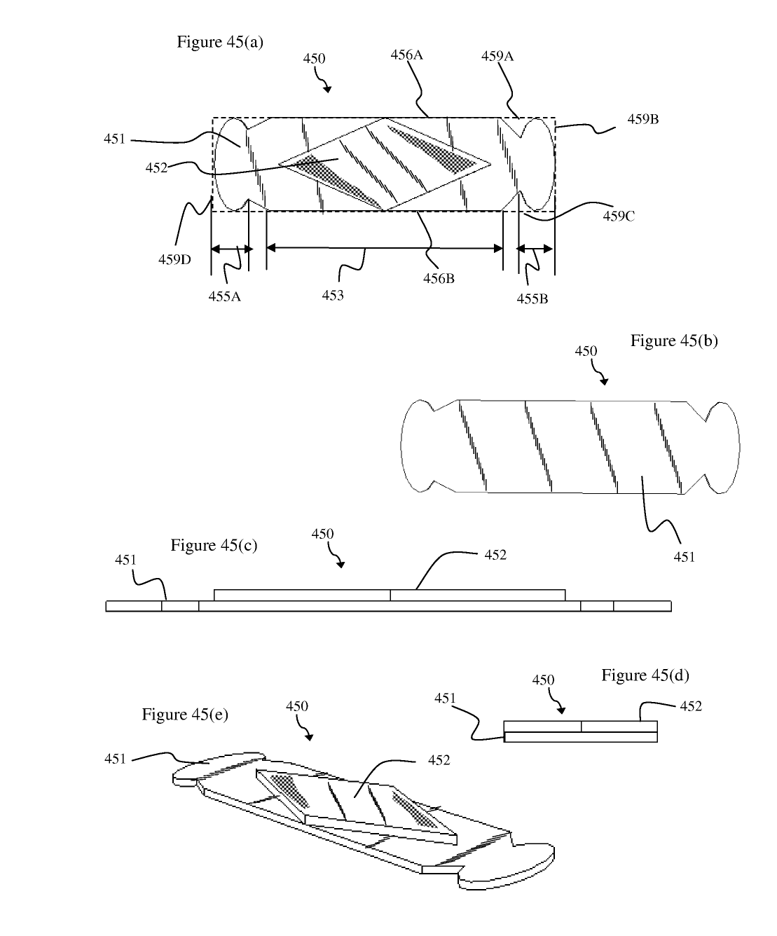

FIGS. 45(a) through 45(e) are, respectively, a bottom wound-facing view, a top non-wound facing view, two side views and one perspective view of an exemplary embodiment of a “diamond gauze” bandage 450 according to the present invention. The “diamond gauze” bandage 450 has a body portion 451 with a central portion 453 that has flattened top and bottom edges 456A, 456B, resulting in a uniform or substantially uniform width for the central portion 453. Oval shaped side regions 455A and 455B connect to the central portion 453 via narrow portions 454A and 454B. The narrow portions 454A and 454B preferably narrow in straight or substantially straight lines from a wider end connected to the central portion 453 and a narrower end connected to the oval shaped side regions 455A and 455B. A gauze pad 452 is oriented with respect to the body portion 451 such that the sides of the gauze pad 452 are not parallel to length tangents 459A, 459C and width tangents 459B, 459D. The gauze pad 452 may have the same center point as the body portion 451 and/or each of the corners of the gauze pad 452 may be oriented toward a mid-point of one of the length tangents 459A, 459C or width tangents 459B, 459D. The central portion 453 is preferably positioned to correspond with the midpoints of the length tangents 459A, 459C.

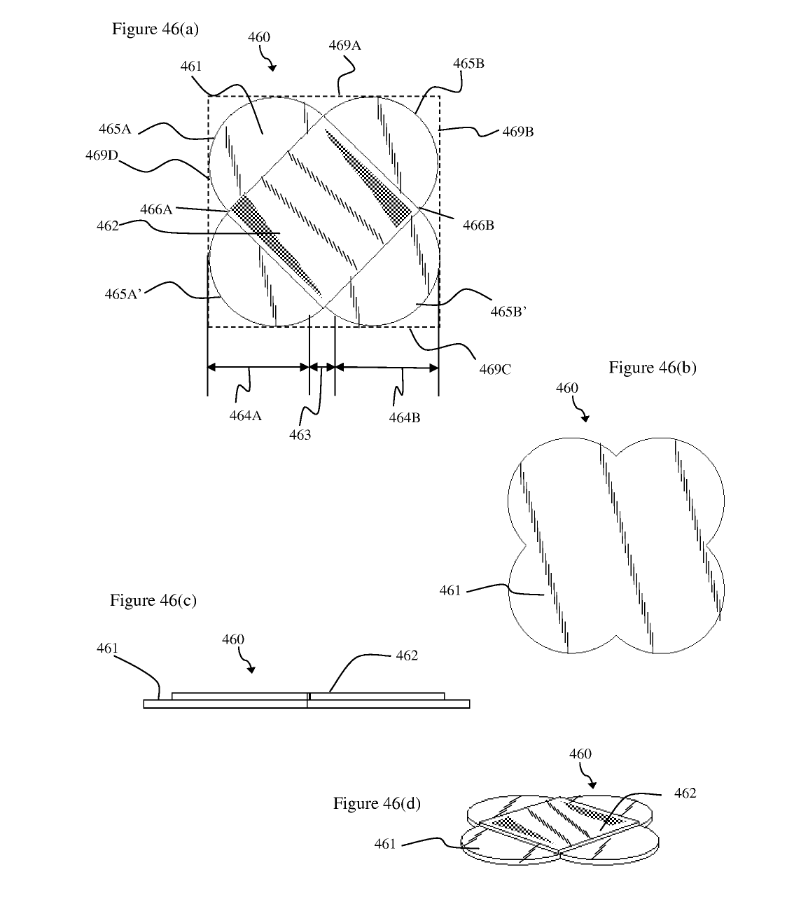

FIGS. 46(a)-46(d) are, respectively, a bottom wound-facing view, a top non-wound facing view, one side view and one perspective view of another exemplary embodiment of a “diamond gauze” bandage 460 according to the present invention. A body portion 461 of the “diamond gauze” bandage 460 has a central narrow portion 463 and side regions 464A, 464B. Side region 464A has edges defined by two circular or elliptical arcs 465A, 465A′ that extend from opposing sides of the central narrow portion 463 and meet at an inflection point 466A. Side region 464B has edges defined by two circular or elliptical arcs 465B, 465B′ that extend from opposing sides of the narrow portion 463 and meet at an inflection point 466B. The gauze pad 462 is oriented with respect to the body portion 461 such that the sides of the gauze pad 462 are not parallel to length tangents 469A, 469C and width tangents 469B, 469D. The gauze pad 462 may have the same center point as the body portion 461 and/or each of the corners of the gauze pad 462 may be oriented toward a mid-point of one of the length tangents 469A, 469C or width tangents 469B, 469D. The inflection points 466A, 466B preferably correspond to midpoints of the width tangents 469D, 469B, respectively. The circular or elliptical arcs 465A, 465A′, 465B, 465B′, are preferably equivalently or similarly sized, and are preferably oriented at a 90 degree angle in relation to adjacent arcs. The central narrow portion 463 is preferably positioned to correspond with the midpoints of the length tangents 469A, 469C.

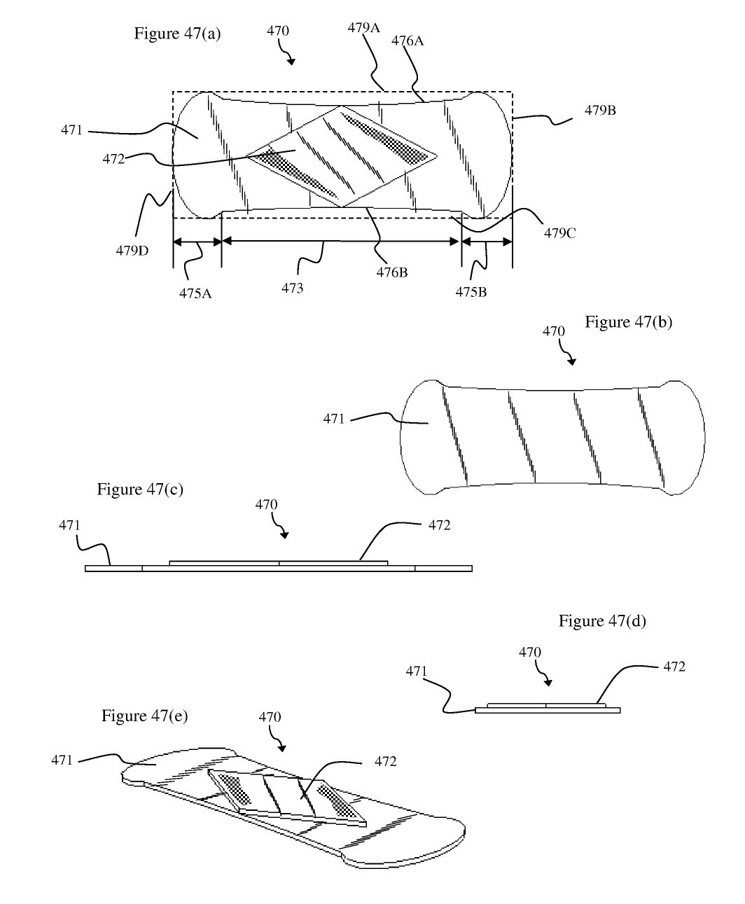

FIGS. 47(a) through 47(e) are, respectively, a bottom wound-facing view, a top non-wound facing view, two side views and one perspective view of an exemplary embodiment of a “diamond gauze” bandage 470 according to the present invention. The “diamond gauze” bandage 470 has a body portion 471 with a central portion 473 that has top and bottom edges 476A, 476B and oval or bulbous shaped side regions 475A, 475B. The “diamond gauze” bandage 470 has a width that is defined by the distance between the top edge 476A and the bottom edge 476B. The top and bottom edges 476A, 476B may be substantially straight, resulting in a uniform or substantially uniform width for the central portion 473. Alternatively, the top and bottom edges 476A, 476B may be concave edges, resulting in a widening width from the middle of the central portion 473 to the oval shaped side regions 475A, 475B. The oval shaped side regions 475A, 475B have a width that is wider than the widest width of the central portion 473. A gauze pad 472 is oriented with respect to the body portion 471 such that the sides of the gauze pad 472 are not parallel to length tangents 479A, 479C and width tangents 479B, 479D. The gauze pad 472 may have the same center point as the body portion 471 and/or each of the corners of the gauze pad 472 may be oriented toward a mid-point of one of the length tangents 479A, 479C or width tangents 479B, 479D. The central portion 473 is preferably positioned to correspond with the midpoints of the length tangents 479A, 479C.

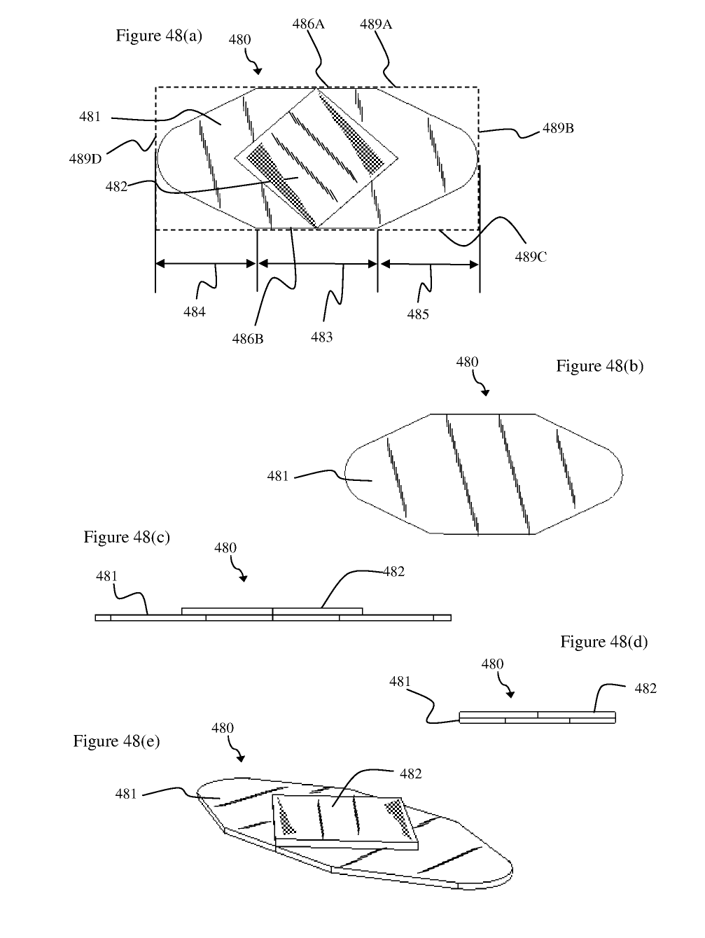

FIGS. 48(a)-48(e) are, respectively, a bottom wound-facing view, a top non-wound facing view, two side views and one perspective view of another exemplary embodiment of a “diamond gauze” bandage 480 according to the present invention. A body portion 481 of the “diamond gauze” bandage 480 has a central portion 483 that has flattened top and bottom edges 486A, 486B, resulting in a uniform or substantially uniform width for the central portion 483. The body portion 481 also has side regions 484 and 485 that, moving along the length from the central portion 483, narrow in straight or substantially lines to rounded ends, where the central portion 483 transitions to the side regions 484 and 485 by sharp angles. The gauze pad 482 is oriented with respect to the body portion 481 such that the sides of the gauze pad 482 are not parallel to length tangents 489A, 489C and width tangents 489B, 489D. The gauze pad 482 may have the same center point as the body portion 481 and/or each of the corners of the gauze pad 482 may be oriented toward a mid-point of one of the length tangents 489A, 489C or width tangents 489B, 489D. Midpoints of the central portion 483 are preferably positioned to correspond with the midpoints of the length tangents 489A, 489C.

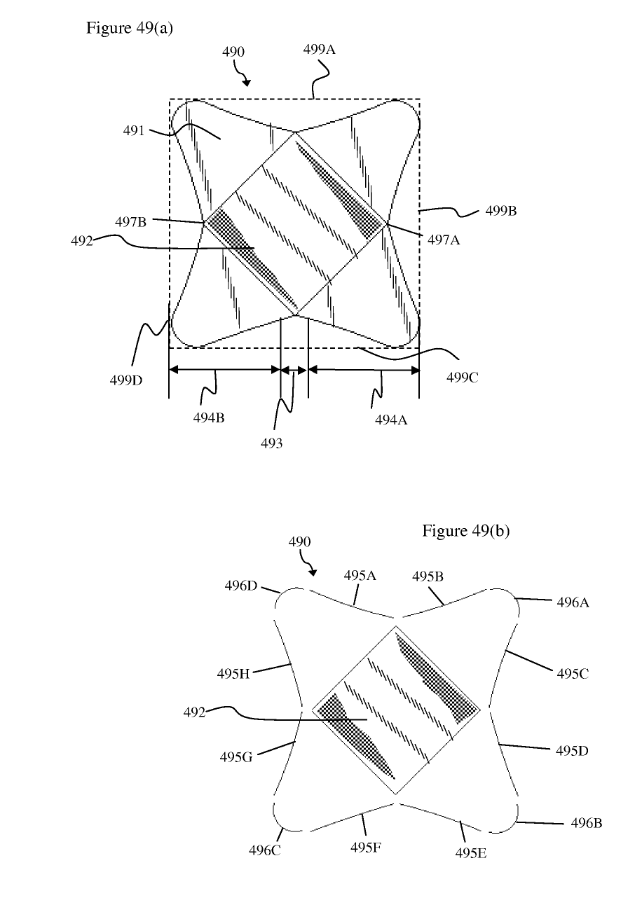

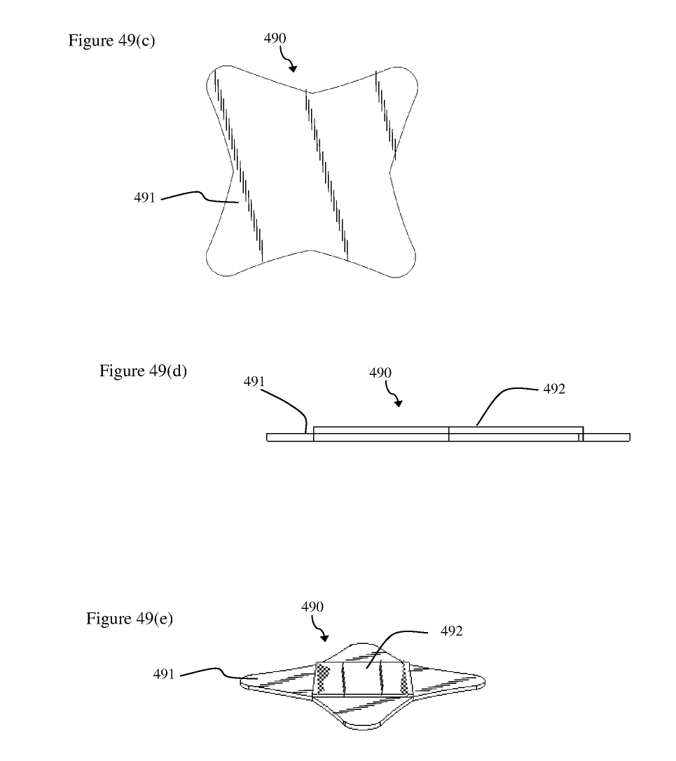

FIGS. 49(a)-49(f) are, respectively, a bottom wound-facing view, an exploded bottom wound-facing view, a top non-wound facing view, two side views and one perspective view of another exemplary embodiment of a “diamond gauze” bandage 490 according to the present invention. A body portion 491 of the “diamond gauze” bandage 490 has a central narrow portion 493 and side regions 494A, 494B that widen with straight, substantially straight, or slightly curved side lines 495A, 495B, 495E, 495F as the side regions 494A, 494B extend to rounded corners 496A, 496B, 496C, 496D. The rounded corners 496A, 496B, 496C, 496D transition to straight, substantially straight, or slightly curved end lines 495C, 495D, 495G, 495H that are oriented so as to taper or decrease a length of the body portion 491 as the end lines 495C, 495D, 495G, 495H extend toward inflection points 497A, 497B. The inflection points 497A, 497B are preferably at midpoints between corresponding rounded corners 496A, 496B, 496C, 496D. The side lines 495A, 495B, 495E, 495F are preferably equal in length and shape to the end lines 495H, 495C, 495D, 495G, respectively. The gauze pad 492 is oriented with respect to the body portion 491 such that the sides of the gauze pad 492 are not parallel to length tangents 499A, 499C and width tangents 499B, 499D. The gauze pad 492 may have the same center point as the body portion 491 and/or each of the corners of the gauze pad 492 may be oriented toward a mid-point of one of the length tangents 499A, 499C or width tangents 499B, 499D. The central narrow portion 493 is preferably positioned to correspond with the midpoints of the length tangents 499A, 499C.

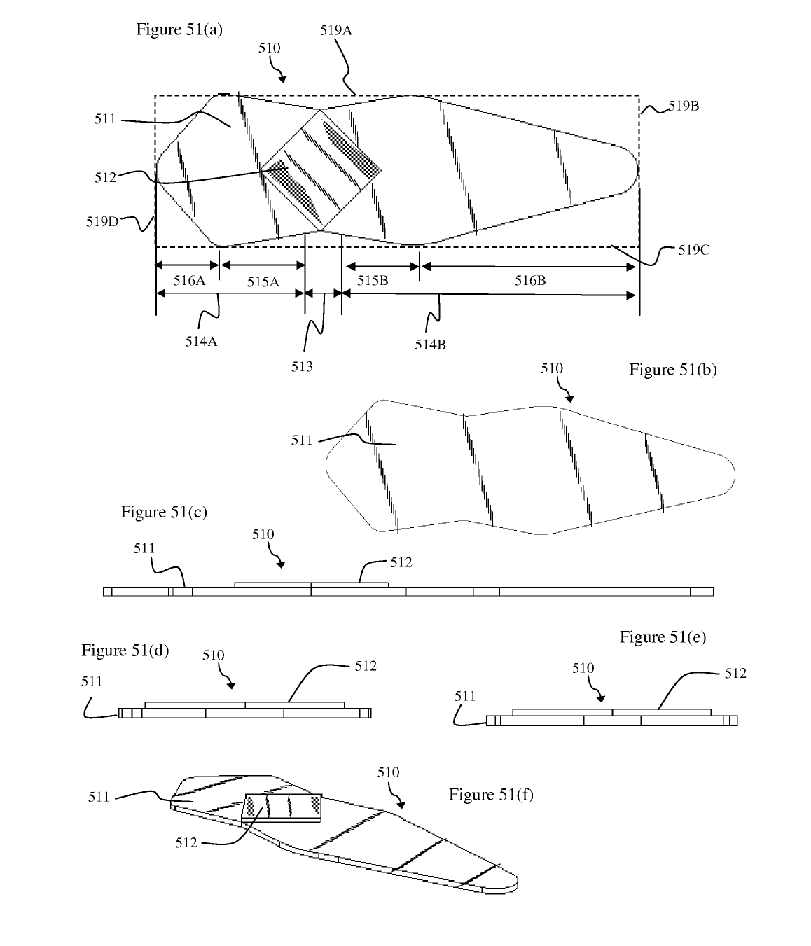

FIGS. 51(a)-51(f) are, respectively, a bottom wound-facing view, a top non-wound facing view, three side views and one perspective view of another exemplary embodiment of a “diamond gauze” bandage 510 according to the present invention. A body portion 511 of the “diamond gauze” bandage 510 has a central narrow portion 513 and side regions 514A, 514B that have widening portions 515A, 515B, that extend from the central narrow portion 513 to narrowing portions 516A, 516B in straight or substantially straight lines. The narrowing portions 516A, 516B extend to rounded ends in straight or substantially straight lines. The widening portions 515A, 515B are connected to the narrowing portions 516A, 516B by rounded angles. The body portion 511 has a “bowtie” or “hourglass” shape with the narrowing portion 516B having a greater length than the narrowing portion 516A. The gauze pad 512 is oriented with respect to the body portion 511 such that the sides of the gauze pad 512 are not parallel to length tangents 519A, 519C and width tangents 519B, 519D. The corners of the gauze pad 512 oriented toward the width tangents 519B, 519D may be oriented toward the mid-points of the width tangents 519B, 519D. The corners of the gauze pad 512 oriented toward the length tangents 519A, 519C may be oriented toward a midpoint between the rounded angles that connect the widening portions 515A, 515B with the narrowing portions 516A, 516B. Due to the asymmetrical lengths of the side regions 514A, 514B, a midpoint of the central narrow portion 513 does not correspond to the midpoint of the length tangents 519A, 519C.

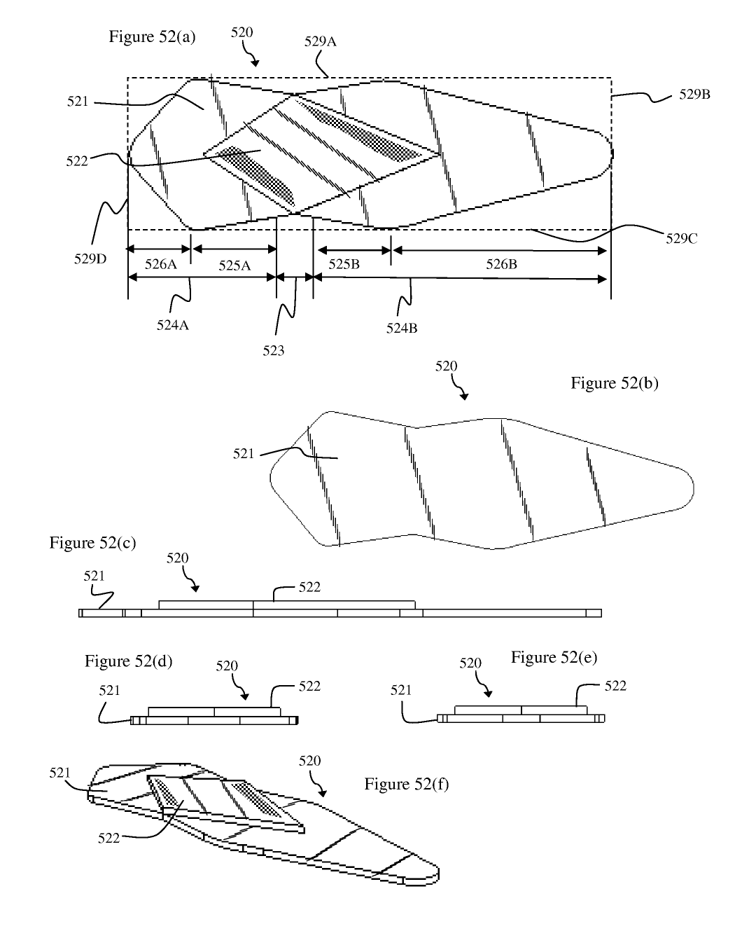

FIGS. 52(a)-52(f) are, respectively, a bottom wound-facing view, a top non-wound facing view, three side views and one perspective view of another exemplary embodiment of a “diamond gauze” bandage 520 according to the present invention. A body portion 521 of the “diamond gauze” bandage 520 is similar to the configuration of the body portion 511 in FIG. 51. The body portion 521 of the “diamond gauze” bandage 520 has a central narrow portion 523 and side regions 524A, 524B that have widening regions 525A, 525B, that extend from the central narrow portion 523 to narrowing portions 526A, 526B in straight or substantially straight lines. The narrowing portions 526A, 526B extend to rounded ends in straight or substantially straight lines. The widening portions 525A, 525B are connected to the narrowing portions 526A, 526B by rounded angles. The body portion 521 has a “bowtie” or “hourglass” shape with the narrowing portion 526B having a greater length than the narrowing portion 526A. The gauze pad 522 is oriented with respect to the body portion 521 such that the sides of the gauze pad 522 are not parallel to length tangents 529A, 529C and width tangents 529B, 529D. The corners of the gauze pad 522 oriented toward the width tangents 529B, 529D may be oriented toward the mid-points of the width tangents 529B, 529D. Due to the asymmetrical lengths of the side regions 524A, 524B, a midpoint of the central narrow portion 523 does not correspond to the midpoint of the length tangents 529A, 529C. However, the corners of the gauze pad 522 oriented toward the length tangents 529A, 529C may be oriented toward a midpoint between the rounded angles that connect the widening portions 525A, 525B with the narrowing portions 526A, 526B. In contrast to the shape of the gauze pad 512 for bandage 510, which has equal sides, the gauze pad 522 for bandage 520 has a “kite-shape”. The “kite shape” may be formed by configuring the sides of the gauze pad 522 that correspond or extend into the side region 524B to be “stretched” to have an increased length corresponding to the increased length in the narrowing portion 526B relative to the length of narrowing portion 526A. Moreover, a kite-shaped gauze pad and/or an increase in length for a single side region may be applied to any “diamond gauze” bandage such as the other exemplary embodiments of a “diamond gauze” bandage described above.

According to the present invention, an alternative to the “diamond gauze” configuration of a bandage is a “triangular gauze” bandage, as discussed below. The “triangular gauze” configuration provides similar advantages to the “diamond gauze” particularly superior adherence and conformability to a wound area with a maximal area of gauze. The “triangular gauze” configuration may be especially useful in bandaging smaller wounds.

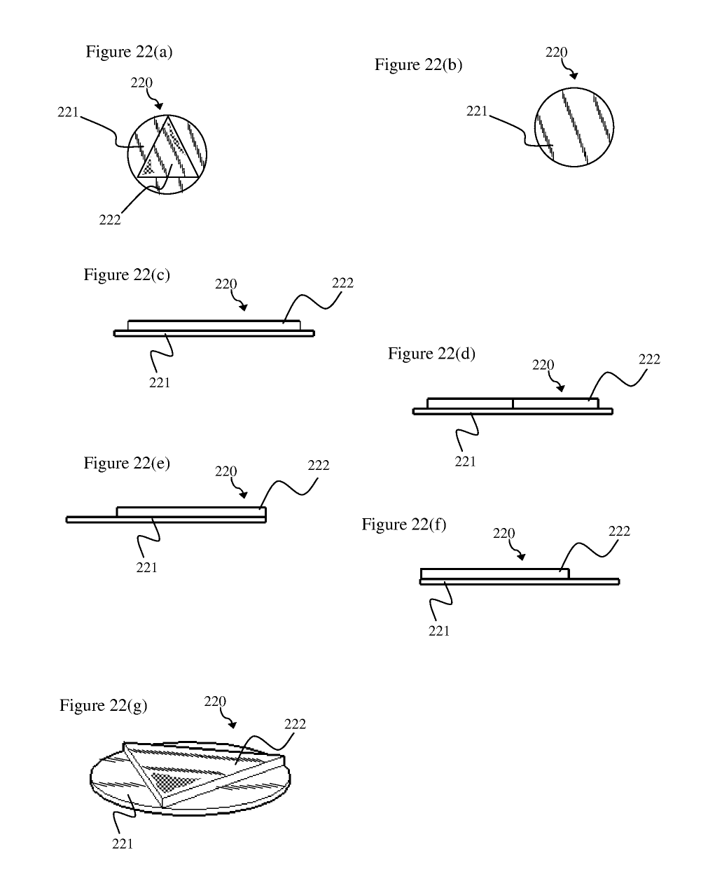

FIGS. 22(a)-22(g) are, respectively, a bottom wound-facing view, a top non-wound facing view, four side views and one perspective view of an exemplary embodiment of a “triangular gauze” bandage 220 according to the present invention. The “triangular gauze” bandage 220 has a body portion 221 in a circular shape, which may be made of any suitable material, preferably a stretchable and breathable material, which is known in the art to be used for bandages. A gauze pad 222 is attached to the wound-facing side of the body portion 221. The gauze pad 222 is a triangular shape, preferably an equilateral triangular shape, but the gauze pad 222 may alternatively be configured as an isosceles, right or other triangular shape and may have rounded or flattened corners. Moreover, just as in the “diamond gauze” bandages, the corners may be rounded and the sides need not be straight but may, for example, be concave or convex curves or wavy lines. The gauze pad 222 may be positioned so as to have the same center point as the body portion 221, with each of the corners of the gauze pad 222 extending to the edges of body portion 221. Alternatively, the gauze pad 222 may be sized so that the corners of the gauze pad 222 do not reach the edges of the body portion 221. The gauze pad 222 may be sized and positioned so as to substantially define three equal quadrants, on the “non-gauze portion” of the wound facing side of the body portion 221, i.e. on the wound facing side of the body portion 221 that is not covered by the gauze pad 222. The bottom wound-facing side of the body portion 221 of the “triangular gauze” bandage 220 may have adhesive as those used for bandages in the art. The adhesive may be applied to all or part of the bottom wound-facing side of the body portion 221 of the “triangular gauze” bandage 220. The gauze pad 222 may or may not be affixed to the body portion 221 of the “triangular gauze” bandage 220 via the same adhesive as that which is used to affix the “triangular gauze” bandage 220 to a patient.

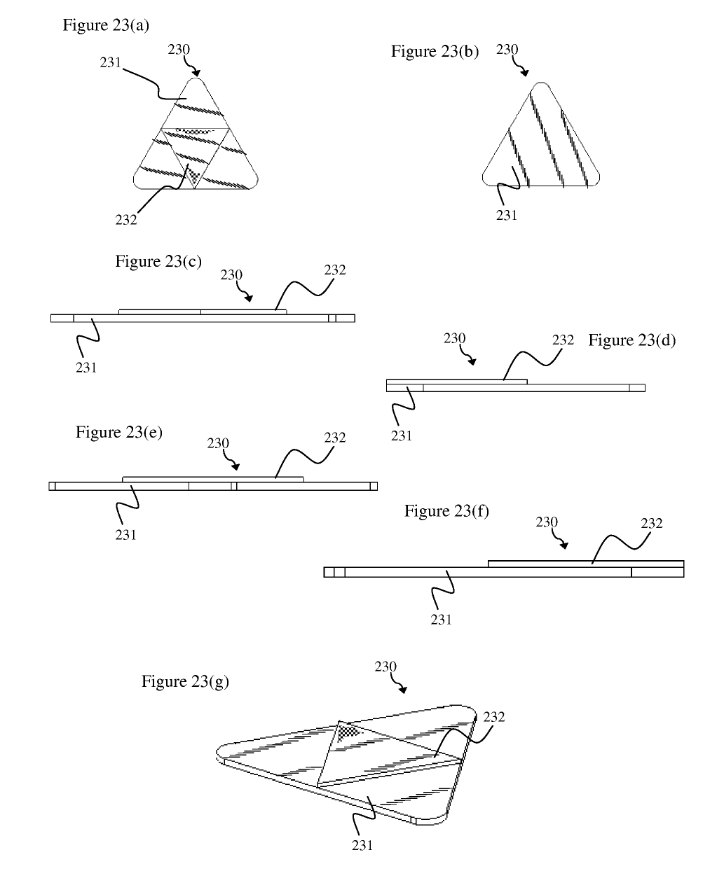

FIGS. 23(a)-23(g) are, respectively, a bottom wound-facing view, a top non-wound facing view, four side views and one perspective view of another exemplary embodiment of a “triangular gauze” bandage 230 according to the present invention. The “triangular gauze” bandage 230 has a body portion 231 in a triangular shape, preferably with rounded corners, and may be made of any suitable material, preferably a stretchable and breathable material, which is known in the art to be used for bandages. The gauze pad 232 is a triangular shape, preferably an equilateral triangular shape, but the gauze pad 232 may alternatively be configured as an isosceles, right or other triangular shape and may have rounded or flattened corners. The gauze pad 232 may be positioned so as to have the same center point as the body portion 231, with each of the corners of the gauze pad 232 extending to the edges of body portion 231. Alternatively, the gauze pad 232 may be sized so that the corners of the gauze pad 232 do not reach the edges of the body portion 231. The gauze pad 232 may be sized and positioned so as to substantially define three equal quadrants on the wound facing side of the body portion 231. The bottom wound-facing side of the body portion 231 of the “triangular gauze” bandage 230 may have adhesive as those used for bandages in the art. The adhesive may be applied to all or part of the bottom wound-facing side of the body portion 231 of the “triangular gauze” bandage 230. The gauze pad 232 may or may not be affixed to the body portion 231 of the “triangular gauze” bandage 230 via the same adhesive as that which is used to affix the “triangular gauze” bandage 230 to a patient.

The embodiments of the invention described herein are exemplary in nature, and therefore, the spirit and the scope of the invention are by no means restricted to what is described above or intended to represent every possible embodiment of the invention. Likewise, structural limitations discussed by one exemplary embodiment of the bandage according to the present invention may be applied to other embodiments and exemplary embodiments of the bandage according to the present invention.

What is claimed is:

1. A bandage comprising
a. a body portion; and
b. a gauze pad on the wound-facing side of the body portion shaped and positioned in such manner that the bandage is configured as a diamond gauze adhesive bandage.

2. The bandage of claim 1, wherein the gauze pad is shaped as a square, rhombus or parallelogram and positioned on the wound-facing side of the body portion in such manner that each of the corners of the gauze pad is oriented towards a different length tangent or width tangent of the body portion.

3. A bandage comprising
a. a body portion; and
b. a gauze pad with three primary corners oriented on a wound-facing side of the body portion.

4. The bandage of claim 3, wherein
a. the body portion is triangular shaped with a perimeter defined by three edges; and
b. the gauze pad is oriented on a wound-facing side of the body portion in such manner that each of the three primary corners of the gauze pad is oriented toward a different edge of the body portion.

5. The bandage of claim 4, wherein the triangular shaped gauze pad is oriented on the wound-facing side of the body portion in such manner that each of the primary corners of the gauze pad is oriented toward a mid-point or a mid-section of a different edge of the body portion, the mid-section being a portion of a corresponding edge equal to half the length of the corresponding edge and centered around the midpoint of the corresponding edge.

6. The bandage of claim 1, wherein the gauze pad is sized and positioned so as to define or substantially define four equal quadrants on a non-gauze portion of a wound facing side of the body portion.

7. The bandage of claim 1, wherein the body portion has adhesive on at least a portion of a wound facing side.

8. The bandage of claim 2, wherein the body portion has adhesive on at least a portion of a wound facing side.

9. The bandage of claim 3, wherein the body portion has adhesive on at least a portion of a wound facing side.

10. The bandage of claim 4, wherein the body portion has adhesive on at least a portion of a wound facing side.

11. The bandage of claim 1, wherein the gauze pad has four sides sized and positioned so as to define or substantially define two equal quadrants on a non-gauze portion of the wound facing side of the body portion, with two sides of the four sides of the gauze pad defining an edge of a first quadrant of the two equal quadrants and another two sides of the four sides of the gauze pad defining an edge of a second quadrant of the two equal quadrants.

12. The bandage of claim 1, wherein the body portion has four tangents, a first and a second width tangents of the four tangents being tangential to opposing ends of the body portion, and a first and a second length tangent of the four tangents being tangential to the edges of the body portion, each of the four tangents having a mid-section, the mid-section equal to half the length of the tangent centered around the midpoint of the tangent, and the gauze pad has four primary corners positioned on the wound-facing side of the body portion in such manner that a first primary corner of the four primary corners is oriented towards a mid-section of a first tangent of the four tangents of the body portion and a second primary corner of the four primary corners that is opposing the first primary corner is oriented towards a mid-section of a second tangent of the four tangents of the body portion.

13. The bandage of claim 3, wherein the body portion is circular shaped.

14. The bandage of claim 12, wherein a third primary corner of the four primary corners of the gauze pad is oriented towards a mid-section of a third tangent of the four tangents of the body portion and a fourth primary corner of the four primary corners of the gauze pad is oriented towards a mid-section of a fourth tangent of the four tangents of the body portion.

15. The bandage of claim 12, wherein the body portion has a central narrow portion with a narrowed width and two side regions, each of the two side regions having a first side, the first sides of the two side regions being on opposing adjacent sides of the central narrow portion and each of the two side regions having a second side, the second sides of the two side regions being on opposing ends of the body portion, each of the side regions having a first side region portion that extends from the first side of the side region toward the second side of the side region, and a width of the first side region portion of each of the side regions widening with respect to the width of the central narrow portion as it extends from the first side of the side regions toward the second side of the side regions.

16. The bandage of claim 15, wherein each of the side regions has a second side region portion that extends from the first side region portion to the second side of the side regions, the width of the second side region portion narrowing as it extends from the first side region portion to the second side of the side region.

17. The bandage of claim 15, wherein the primary corners of the gauze pad that are oriented toward the length tangents are located within the central narrow portion.

18. The bandage of claim 17, wherein a midpoint of the central narrow portion does not correspond with a midpoint of the length tangents.

19. The bandage of claim 18, wherein the gauze pad is configured with a kite-shape.

20. The bandage of claim 12, wherein the body portion has a central portion and two legs that extend in a lengthwise direction from the central portion toward the same width tangent.

21. The bandage of 20, wherein the body portion forms an arch shape defined by two parallel arcs or parabolas that are connected to each other at respective proximate ends distal to the central portion.

22. A bandage comprising
a. a bowtie shaped body portion having a wound-facing side; and
b. a rectangular or square gauze pad positioned on the wound-facing side of the body portion in such manner that sides of the gauze pad are parallel to a length tangent or a width tangent of the body portion.

23. The bandage of claim 15, wherein the edges at the opposing ends of the body portion are defined by end lines that are oriented so as to taper the length of the body portion as the end lines extend from corners of the body portion toward an inflection point along each of the opposing ends.

24. The bandage of claim 12, wherein the body portion has a central almond or oval shaped portion and bulbous side portions extending from opposing sides of the central almond or oval shaped portion.

25. The bandage of claim 15, wherein an edge of each of the side regions is defined by a single circular or elliptical arc.

26. The bandage of claim 15, wherein an edge of each of the side regions is defined by two circular or elliptical arcs that extend from opposing sides of the central narrow portion and meet at an inflection point.

27. The bandage of claim 12, wherein the body portion has a central portion that has a uniform or substantially uniform width defined by flattened top and bottom edges and two side regions extending from opposing sides of the central portion to opposing ends of the body portion, the width of the side regions narrowing as the side regions extend from the central portion to the ends of the body portion.

28. The bandage of claim 14, wherein the body portion is square or rectangular shaped.

29. The bandage of claim 14, wherein a center point of the gauze pad coincides with a center point of the body portion and two opposing primary corners of the gauze pad extend to the edges of the body portion.

30. The bandage of claim 14, wherein the body portion is square or rectangular shaped with rounded corners and the gauze pad is shaped as a square, rhombus or parallelogram.

31. The bandage of claim 14, wherein the body portion is almond or oval shaped.

32. The bandage of claim 12, wherein the body portion is comprised of a head portion and a tail portion, the head portion has a truncated oval, diamond, or almond shape, and the tail portion flares out with a width that increases as the tail extends lengthwise from a first side of the head portion forming a concavity with the head portion along a top edge and a bottom edge of the body portion.

33. The bandage of claim 12, wherein a first end region of the body portion extends to a first end of the body portion proximal to the first width tangent of the body portion and a second end region of the body portion extends to a second end of the body portion proximal to the second width tangent, the second end region having a smaller width than the first end region.

34. The bandage of claim 33, wherein a width of the body portion reduces or remains the same as it extends from the first end region to the second end region.

35. The bandage of claim 34, wherein the second side region has a greater length than the first side region and a top edge and a bottom edge of the body portion are each configured as a continuous convex curve.

36. A bandage comprising
a. a body portion having a head portion and a tail portion, the head portion having a truncated oval, diamond, or almond shape, and the tail portion flaring out with a width that increases as the tail extends lengthwise from a first side of the head portion forming a concavity with the head portion along a top edge and a bottom edge of the body portion; and
b. a gauze pad with four sides positioned on the wound-facing side of the body portion in such manner that each of the four sides are parallel to a length tangent or a width tangent of the body portion.

37. The bandage of claim 12, wherein the body portion has a central portion and oval shaped side regions extending from opposing sides of the central portion.

38. The bandage of claim 37, wherein the central portion has a uniform or substantially uniform width defined by flattened top and bottom edges.

39. The bandage of claim 38, wherein the body portion has narrow portions that connect the central portion to each of the oval shaped side regions.

40. The bandage of claim 37, wherein the central portion has concave top and bottom edges, resulting in a widening width from the middle of the central portion to the oval shaped end portions.

41. The bandage of claim 1, wherein the gauze pad has four primary corners positioned on the wound-facing side of the body portion in such manner that any line drawn between any two adjacent primary corners of the gauze pad is not parallel to any length tangent or width tangent of the body portion.

42. The bandage of claim 41, wherein a ray of each primary corner intersects one of the length tangents or width tangents of the body portion at an angle between 55 and 90 degrees.

43. The bandage of claim 41, wherein a ray of each primary corner intersects one of the length tangents or width tangents of the body portion at an angle between 65 and 90 degrees.

44. The bandage of claim 41, wherein a ray of each of at least two primary corners intersects a length tangent or width tangent of the body portion at a point in a mid-section of the length tangent or width tangent, the mid-section being a portion of a corresponding length tangent or width tangent equal to half the length of the corresponding length tangent or width tangent and centered around the midpoint of the corresponding length tangent or width tangent.
